(19) United States
(12) Patent Application Publication
(10) Pub. No.: US 2008/0207483 A1
(43) Pub. Date: Aug. 28, 2008

(54) Methods and compositions for diagnosis and therapy of cancer based on hyper-mutated tumor genes

(75) Inventor: Stefano Volinia, Ferrara (IT)

Correspondence Address: STEFANO VOLINIA, CORSO PIAVE 17, FERRARA, 44100, IT

(73) Assignee: Dr. Stefano Volinia, Ferrara (IT)

(21) Appl. No.: 11/333,076

(22) Filed: Jan. 17, 2006

Related U.S. Application Data
(60) Provisional application No. 60/593,456, filed on Jan. 16, 2005.

(51) Int. Cl. A61K 38/02; C12Q 1/68; C40B 30/04

(52) U.S. Cl. 514/2; 435/6; 506/9

(57) ABSTRACT

The present invention provides novel methods and compositions for the diagnosis and treatment of cancers. The invention also provides methods of identifying inhibitors of tumorigenesis.

### BACKGROUND OF THE INVENTION

Cancer, the uncontrolled growth of malignant cells, is a major health problem of the modern medical era and is one of the leading causes of death in developed countries. In the United States, one in four deaths is caused by cancer (Jemal, A. et al., CA Cancer J. Clin. 52:23-47 (2002)). Among cancers, those that arise from organs and solid tissues, known as cancers (e.g., colon cancer, lung cancer, breast cancer, stomach cancer, prostate cancer, pancreatic cancer) are among the most-commonly identified human cancers.

For example, prostate cancer is the most frequently diagnosed noncutaneous malignancy among men in industrialized countries, and, in the United States, 1 in 8 men will develop prostate cancer during his life (Simard, J. et al., Endocrinology 743(6):2029-40 (2002)). The incidence of prostate cancer has dramatically increased over the last decades and prostate cancer is now a leading cause of death in the United States and Western Europe (Peschel, R. E. and J. W. Colberg, Lancet 4:233-41 (2003); Nelson, W. G. et al., N. Engl. J. Med. 349(4):366-81 (2003)). An average 40% reduction in life expectancy affects males with prostate cancer. If detected early, prior to metastasis and local spread beyond the capsule, prostate cancer can often times be cured (e.g., using surgery). However, if diagnosed after spread and metastasis from the prostate, prostate cancer is typically a fatal disease with low cure rates. While prostate-specific antigen (PSA)-based screening has aided early diagnosis of prostate cancer, it is neither highly sensitive nor specific (Punglia et. al., N Engl J Med. 349(4):335-42 (2003)). This means that a high percentage of false negative and false positive diagnoses are associated with the test. The consequences are both many instances of missed cancers and unnecessary follow-up biopsies for those without cancer.

Breast cancer remains the second leading cause of cancer-related deaths in women, affecting more than 180,000 women in the United States each year. For women in North America, the life-time odds of getting breast cancer are now one in eight. Although the discovery of BRCA1 and BRCA2 were important steps in identifying key genetic factors involved in breast cancer, it has become clear that mutations in BRCA1 and BRCA2 account for only a fraction of inherited susceptibility to breast cancer (Nathanson, K. L. et al., Human Mol. Gen. 70(7):715-720 (2001); Anglican Breast Cancer Study Group. Br. J. Cancer 83(10): 1301-08 (2000); and Syrjakoski K., et al., J. Natl. Cancer Inst. 92:1529-31 (2000)). Despite considerable research into therapies for breast cancer, breast cancer remains difficult to diagnose and treat effectively, and the high mortality observed in breast cancer patients indicates that improvements are needed in the diagnosis, treatment and prevention of the disease.

Excluding skin cancer, colorectal cancer is the third most frequently diagnosed cancer in the United States and Canada (after lung and breast in women, and lung and prostate in men). The American Cancer Society estimates that there will be approximately 145,000 new cases of colorectal cancer diagnosed in the U.S. in 2005 (Cancer Facts and Figures 2005. Atlanta, Ga.: American Cancer Society, 2005. Available at www.cancer.org/docroot/STT/stt_{—}0.asp, accessed Dec. 19, 2005). Colorectal cancer is the second leading cause of cancer death among men and women in the United States and Canada (after lung cancer).

The annual incidence of pancreatic cancer is nearly equivalent to the annual mortality, estimated to be 31,860 and 31,270, respectively, in the U.S. in 2004 (Cancer Facts and Figures 2004. Atlanta, Ga.: American Cancer Society, 2004. Available at www.cancer.org/docroot/STT/stt_{—}0_{—}2004.asp, accessed Aug. 21, 2005). Patients with locally advanced and metastatic pancreatic cancer have poor prognoses, and diagnosis generally occurs too late for surgery or radiotherapy to be curative (Burr, H. A., et al., The Oncologist 10(3): 183-190, (2005)). Chemotherapy can provide relief of symptoms for some patients with advanced pancreatic cancer, but its impact on survival has been modest to date.

In the United States, more than 20,000 individuals are diagnosed with stomach (gastric) cancer each year. The American Cancer Society estimates that there will be 22,710 new cases of colorectal cancer diagnosed in the U.S. in 2004 (Cancer Facts and Figures 2004. Atlanta, Ga.: American Cancer Society, 2004. Available at www.cancer.org/docroot/STT/stt_{—}0_{—}2004.asp, accessed Aug. 21, 2005). Because stomach cancer may occur without symptoms, it may be in advanced stages by the time the diagnosis is made. Treatment is then directed at making the patient more comfortable and improving quality of life.

Lung cancer causes more deaths worldwide than any other form of cancer (Goodman, G. E., Thorax 57994-999 (2002)). In the United States, lung cancer is the primary cause of cancer death among both men and women. In 2002, the death rate from lung cancer was an estimated 134,900 deaths, exceeding the combined total for breast, prostate and colon cancer. Id. Lung cancer is also the leading cause of cancer death in all European countries, and numbers of lung cancer-related deaths are rapidly increasing in developing countries as well.

The five-year survival rate among all lung cancer patients, regardless of the stage of disease at diagnosis, is only about 13%. This contrasts with a five-year survival rate of 46% among cases detected while the disease is still localized. However, only 16% of lung cancers are discovered before the disease has spread. Early detection is difficult as clinical symptoms are often not observed until the disease has reached an advanced stage. Despite research into therapies for this and other cancers, lung cancer remains difficult to diagnose and treat effectively. Clearly, the identification of markers and genes that are responsible for susceptibility to particular forms of solid cancer (e.g., prostate cancer, breast cancer, lung cancer, stomach cancer, colon cancer, pancreatic cancer) is one of the major challenges facing oncology today. There is a need to identify means for the early detection of individuals that have a genetic susceptibility to cancer so that more aggressive screening and intervention regimens may be instituted for the early detection and treatment of cancer. Cancer genes may also reveal key molecular pathways that may be manipulated (e.g., using small or large molecule weight drugs) and may lead to more effective treatments regardless of the cancer stage when a particular cancer is first diagnosed.

A variety of approaches have been applied to the identification of cancer genes. Procedures were developed that have allowed identification of genes causative of cellular transformation (Reddy, E. P., Reynolds, R. K., Santos, E. & Barbacid, M. Nature 300, 149-152 (1982); Tabin, C. J. et al. Nature 300, 143-149 (1982)), and of complex processes such as invasiveness and metastasis (Douma, S. et al. Nature. 430:1034-9 (2004)). In vitro methods, using cellular or animal models, led to the discovery of dominant cancer genes, or oncogenes. Many well-characterised cancer genes harbour somatic base substitutions or small insertion/deletions. For example, coding regions frame-shifts and point mutations account for 75% of the somatic mutations in the two major TP53 tumour suppressor genes (Olivier M. et al. Hum Mutat. 19:607-14 (2002); Stenson P. D. et al. Hum Mutat 21:577-81 (2003)). Large-scale sequencing approaches identified PI3K and some tyrosine phosphatases as somatically mutated in human colorectal cancer (Wang, Z. et al. Science 304:1164-6 (2004)). The B-raf oncogene, first described over 20 years ago, was recently shown to be mutated in human cancer (Garnett, M. & Marais, R. Cancer Cell 16:313-319 (2004)). On the other hand, other cancer genes have been discovered through the phenomenon of inherited predisposition. Familial cancer is rare in comparison to non-hereditary cancer, but a number of recessive, or tumour-suppressor, genes have been identified using linkage analysis (Friend, S. H. et al. Nature 323: 643-646 (1986); Breast Cancer Linkage Consortium. Lancet 349: 1505-1510 (1997)). A number of efforts are currently underway to build integrated databases to enable sequence-based cancer genomics (Strausberg, R., L., Simpson A., J., & Wooster, R. Nat Rev Genet. 4:409-418 (2003)). Recently, Futreal et al (Futreal, P. A. et al. Nat Rev Cancer 4:177-83 (2004)) conducted a census from the literature of the genes that are mutated and causally implicated in cancer development, indicating that 299 genes contribute to human cancer. However 70% of these genes are associated with leukaemia, lymphomas and mesenchymal tumours, which account for only 10% of total cancer incidence. Furthermore, about 75% of genes in the census are associated with translocations and while at least 90% of them are dominant at the cellular level (i.e. activated oncogenes, fusion oncoproteins) 90% of germlne mutations are recessive (Futreal, P. A. et al. Nat Rev Cancer 4:177-83 (2004)). Thus it may be that the majority of cancer genes still remain undiscovered. In particular, for most adult epithelial cancer, the four to seven somatically mutated genes that are usually proposed to be necessary for cancer development have not yet been identified.

Clearly, there is a great need in the art for improved methods for detecting and treating cancers (e.g., prostate cancer, breast cancer, lung cancer, stomach cancer, colon cancer, pancreatic cancer). The present invention provides novel methods and compositions for the diagnosis and treatment of cancers.

### SUMMARY OF THE INVENTION

The present invention is based, in part, on the identification of specific genes that have altered genetic structure (i.e. nucleotide sequence or genomic structure) in particular cancers. A novel and systematic approach was devised and applied it to the identification of the genes that are mutated in naturally occurring human cancer. More than 3×10⁹ nucleotides of human coding sequence were analysed from over 5,600,000 expression sequence tags (ESTs) derived from both healthy and cancer tissues. ESTs represent single alleles and are unverified sequences; therefore the detected mismatches are the sum of true mutations and sequencing artefacts. Previous investigators attempted to evaluate sequencing error rate in ESTs (Irizarry, K. et al. Nat. Genet. 26:233-236 (2000)). Here an alternative strategy was followed, based on the assumption that the error rate is identical in the same gene between and within normal and cancer EST libraries.

Accordingly, the invention encompasses methods of diagnosing whether a subject has, or is at risk for developing, a cancer. According to the methods of the invention, the nucleotide sequence and genomic structure of at least one hypermutated cancer gene product in a test sample from the subject is compared to the nucleotide sequence or genomic structure of a corresponding hypermutated cancer gene product in a control sample. An alteration in the nucleotide sequence or genomic structure of the hypermutated cancer gene product in the test sample, relative to the nucleotide sequence or genomic structure of a corresponding hypermutated cancer gene product in a control sample, is indicative of the subject either having, or being at risk for developing, a cancer. The cancer can be any cancer that arises from organs and tissues. In certain embodiments, the cancer is stomach cancer, breast cancer, pancreatic cancer, colon cancer, lung cancer or prostate cancer. In particular embodiments, the cancer is not breast cancer, lung cancer, prostate cancer, pancreatic cancer or gastrointestinal cancer.

In one embodiment, the at least one cancer gene product assayed in the test sample is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247 and combinations thereof. In another embodiment, the at least one cancer gene product assayed in the test sample is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

The nucleotide sequence and gene structure of the at least one cancer gene product can be assayed using a variety of techniques that are well known to those of skill in the art (e.g., DNA sequencing, cloning, quantitative or semi-quantitative PCR and RT-PCR, Southern blot analysis, Northern blot analysis, Western blot analysis, solution hybridization detection). In a particular embodiment, the nucleotide sequence and structure of at least one cancer gene product is assayed by labeling genomic DNA from a test sample obtained from the subject to provide a set of target oligodeoxynucleotides, hybridizing the target oligodeoxynucleotides to one or more cancer gene-specific probe oligonucleotides (e.g., hybridizing to a microarray that comprises several cancer gene-specific probe oligonucleotides) to provide a hybridization profile for the test sample, and comparing the test sample hybridization profile to a hybridization profile from a control sample. An alteration in the signal of at least one cancer gene in the test sample relative to the control sample is indicative of the subject either having, or being at risk for developing, a cancer.

In another embodiment, the nucleotide sequence and structure of at least one cancer gene product is assayed by reverse transcribing RNA from a test sample obtained from the subject to provide a set of target oligodeoxynucleotides, hybridizing the target oligodeoxynucleotides to one or more cancer gene-specific probe oligonucleotides (e.g., hybridizing to a microarray that comprises several cancer gene-specific probe oligonucleotides) to provide a hybridization profile for the test sample, and comparing the test sample hybridization profile to a hybridization profile from a control sample. An alteration in the signal of at least one cancer gene in the test sample relative to the control sample is indicative of the subject either having, or being at risk for developing, a cancer.

In a particular embodiment, target oligonucleotides are hybridized to a microarray comprising cancer gene-specific probe oligonucleotides for one or more cancer genes selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

The invention also encompasses methods of inhibiting tumorigenesis in a subject who has, or is suspected of having, a cancer (e.g., liver cancer, head and neck cancer, prostate cancer, stomach cancer, pancreatic cancer, lung cancer, breast cancer, colon cancer, lymphoma, leukemia, sarcomas), wherein at least one cancer gene product is altered in its nucleotide sequence or genomic structure in the cancer cells of the subject. When the at least one isolated cancer gene product is altered in the cancer cells, the method comprises administering an effective amount of an isolated cancer gene product, an isolated variant or a biologically-active fragment of the cancer gene product or variant, such that proliferation of cancer cells in the subject is inhibited. In a further embodiment, the at least one isolated cancer gene product is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof. When the at least one isolated cancer gene product is activated in the cancer cells, the method comprises administering to the subject an effective amount of at least one compound for inhibiting activity of the at least one cancer gene product (referred to herein as a “cancer gene activity-inhibition compound”), such that proliferation of cancer cells in the subject is inhibited. In a particular embodiment, the at least one cancer gene activity-inhibition compound is specific for a cancer gene product selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

In a related embodiment, the methods of inhibiting tumorigenesis in a subject additionally comprise the steps of determining the nucleotide sequence or genomic structure of at least one cancer gene product in cancer cells from the subject, and comparing that sequence or structure of the cancer gene product in the cells to the level of a corresponding cancer gene product in control cells. If structure of the cancer gene product is altered in cancer cells, the methods further comprise varying the structure of the at least one cancer gene product expressed in the cancer cells. In one embodiment, the amount of the cancer gene product or activity present in the cancer cells is less than the amount of the cancer gene product or activity present in a control cell (e.g., corresponding normal cells), and an effective amount of the cancer gene product, isolated variant or biologically-active fragment of the cancer gene product or variant, is administered to the subject. Suitable hypermutated cancer gene products for this embodiment include NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof, among others. In a particular embodiment, the hypermutated cancer gene product is not NM_{—}000546 or NM_{—}000314. In another embodiment, the amount of the cancer gene product or activity present in the cancer cells is greater than the amount of the cancer gene product or activity present in the control cell (e.g., normal tissue cells), and an effective amount of at least one compound for inhibiting expression of the at least one altered cancer gene product is administered to the subject. Suitable compounds for inhibiting expression of the at least one hypermutated cancer gene product include, but are not limited to, compounds that inhibit the activity of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

The invention further provides pharmaceutical compositions for treating cancers (e.g., liver cancer, prostate cancer, stomach cancer, pancreatic cancer, lung cancer, breast cancer, colon cancer, lymphoma, leukemia). In one embodiment, the pharmaceutical compositions comprise at least one isolated cancer gene product and a pharmaceutically-acceptable carrier. In a particular embodiment, the at least one cancer gene product corresponds to a cancer gene product that has a nucleotide sequence or gene structure in cancer cells relative to control cells. In certain embodiments the isolated cancer gene product is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

In another embodiment, the pharmaceutical compositions of the invention comprise at least one cancer gene expression or activity-inhibition compound. In a particular embodiment, the at least one cancer gene expression or activity-inhibition compound is specific for a cancer gene product whose expression or activity is greater in cancer cells than in control cells. In certain embodiments, the cancer gene expression or activity-inhibition compound is specific for one or more cancer gene products selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

The invention also encompasses methods of identifying an inhibitor of tumorigenesis, comprising providing a test agent to a cell and measuring the level or activity of at least one cancer gene product in the cell. In one embodiment, the method comprises providing a test agent to a cell and measuring the level of at least one cancer gene product associated with altered gene structure in cancers (e.g., liver cancer, prostate cancer, stomach cancer, pancreatic cancer, lung cancer, breast cancer, colon cancer, sarcomas, lymphomas, leukemias). An increase in the level of the cancer gene product or activity in the cell, relative to a suitable control cell, is indicative of the test agent being an inhibitor of tumorigenesis. In a particular embodiment, the at least one cancer gene product associated with altered gene structure in cancer cells is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

In other embodiments, the method comprises providing a test agent to a cell and measuring the level of at least one cancer gene product associated with altered gene structure or activity levels in cancers. A decrease in the level of the cancer gene product or activity in the cell, relative to a suitable control cell, is indicative of the test agent being an inhibitor of tumorigenesis. In a particular embodiment, the at least one cancer gene product associated with altered gene structure in cancer cells is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

### BRIEF DESCRIPTION OF THE DRAWINGS

FIG. 1 depicts a flow diagram shows the overall procedure for discovery of the mutated human genes in cancer.

FIG. 2 depicts the in silico mutation profile of TP53 point mutation analysis in human cancer ESTs.

FIG. 3 depicts the in silico CGH-like profile of TP53 for detection of deletion and amplification in human cancer ESTs.

### DETAILED DESCRIPTION OF THE INVENTION

The present invention is based, in part, on the identification of particular genes whose structure is altered in cancer cells associated with a variety of different cancers, such as colon, stomach, lung, breast and prostate cancer, lymphomas and leukemias relative to normal control cells.

TABLE 1
Human hypermutated cancer genes.

| | | |
|---|---|---|
| GENBANK | | SEQ ID |
| ID | Hypermutated cancer gene description | NO |

| | | |
|---|---|---|
| NM_001273 | Homo sapiens chromodomain helicase DNA binding protein 4 (CHD4), mRNA. | 1 |
| NM_080921 | Homo sapiens protein tyrosine phosphatase, receptor type, C (PTPRC), transcript | 2 |
| | variant 2, mRNA. | |
| NM_014865 | Homo sapiens chromosome condensation-related SMC-associated protein 1 (CNAP1), | 3 |
| | mRNA. | |
| NM_003072 | Homo sapiens SWI/SNF related, matrix associated, actin dependent regulator of | 4 |
| | chromatin, subfamily a, member 4 (SMARCA4), mRNA. | |
| NM_004104 | Homo sapiens fatty acid synthase (FASN), mRNA. | 5 |
| NM_004990 | Homo sapiens methionine-tRNA synthetase (MARS), mRNA. | 6 |
| NM_004599 | Homo sapiens sterol regulatory element binding transcription factor 2 (SREBF2), | 7 |
| | mRNA. | |
| NM_013417 | Homo sapiens isoleucine-tRNA synthetase (IARS), transcript variant long, mRNA. | 8 |
| NM_172230 | Homo sapiens HRD1 protein (HRD1), transcript variant 2, mRNA. | 9 |
| NM_148842 | Homo sapiens Williams-Beuren syndrome chromosome region 16 (WBSCR16), transcript | 10 |
| | variant 2, mRNA. | |
| NM_021948 | Homo sapiens chondroitin sulfate proteoglycan BEHAB (BCAN), transcript variant 1, | 11 |
| | mRNA. | |
| NM_014014 | Homo sapiens activating signal cointegrator 1 complex subunit 3-like 1 (ASCC3L1), | 12 |
| | mRNA | |
| NM_001417 | Homo sapiens eukaryotic translation initiation factor 4B (EIF4B), mRNA. | 13 |
| NM_002271 | Homo sapiens karyopherin (importin) beta 3 (KPNB3), mRNA. | 14 |
| NM_005030 | Homo sapiens polo-like kinase 1 (Drosophila) (PLK1), mRNA. | 15 |
| NM_182917 | Homo sapiens eukaryotic translation initiation factor 4 gamma, 1 (EIF4G1), transcript | 16 |
| | variant 1, mRNA. | |
| NM_001923 | Homo sapiens damage-specific DNA binding protein 1, 127 kDa (DDB1), mRNA. | 17 |
| NM_005762 | Homo sapiens tripartite motif-containing 28 (TRIM28), mRNA. | 18 |
| NM_005348 | Homo sapiens heat shock 90 kDa protein 1, alpha (HSPCA), mRNA. | 19 |
| NM_001418 | Homo sapiens eukaryotic translation initiation factor 4 gamma, 2 (EIF4G2), mRNA. | 20 |
| NM_002266 | Homo sapiens karyopherin alpha 2 (RAG cohort 1, importin alpha 1) (KPNA2), mRNA. | 21 |
| NM_012218 | Homo sapiens interleukin enhancer binding factor 3, 90 kDa (ILF3), transcript variant | 22 |
| | 1, mRNA. | |
| NM_002466 | Homo sapiens v-myb myeloblastosis viral oncogene homolog (avian)-like 2 (MYBL2), mRNA. | 23 |
| NM_005557 | Homo sapiens keratin 16 (focal non-epidermolytic palmoplantar keratoderma) (KRT16), | 24 |
| | mRNA. | |
| NM_000691 | Homo sapiens aldehyde dehydrogenase 3 family, memberA1 (ALDH3A1), mRNA. | 25 |
| NM_001569 | Homo sapiens interleukin-1 receptor-associated kinase 1 (IRAK1), mRNA. | 26 |
| NM_001090 | Homo sapiens ATP-binding cassette, sub-family F (GCN20), member 1 (ABCF1), mRNA. | 27 |
| NM_201524 | Homo sapiens G protein-coupled receptor 56 (GPR56), transcript variant 2, mRNA. | 28 |
| NM_002291 | Homo sapiens laminin, beta 1 (LAMB1), mRNA. | 29 |
| NM_002230 | Homo sapiens junction plakoglobin (JUP), transcript variant 1, mRNA. | 30 |
| NM_001605 | Homo sapiens alanyl-tRNA synthetase (AARS), mRNA. | 31 |
| NM_017647 | Homo sapiens FtsJ homolog 3 (E. coli) (FTSJ3), mRNA. | 32 |
| NM_002541 | Homo sapiens oxoglutarate (alpha-ketoglutarate) dehydrogenase (lipoamide) (OGDH), | 33 |
| | mRNA. | |
| NM_005438 | Homo sapiens FOS-like antigen 1 (FOSL1), mRNA. | 34 |
| NM_133645 | Homo sapiens mitochondrial translation optimization 1 homolog (S. cerevisiae) | 35 |
| | (MTO1), mRNA. | |
| XM_290401 | Homo sapiens hypothetical protein L0C340318 (L0C340318), mRNA. | 36 |
| NM_000968 | Homo sapiens ribosomal protein L4 (RPL4), mRNA. | 37 |
| NM_144733 | Homo sapiens E1B-55 kDa-associated protein 5 (E1B-AP5), transcript variant 2, mRNA. | 38 |
| NM_004741 | Homo sapiens nucleolar and coiled-body phosphoprotein 1 (NOLC1), mRNA. | 39 |
| NM_020414 | Homo sapiens DEAD (Asp-Glu-Ala-Asp) box polypeptide 24 (DDX24), mRNA. | 40 |
| NM_004793 | Homo sapiens protease, serine, 15 (PRSS15), nuclear gene encoding mitochondrial | 41 |
| | protein, mRNA. | |
| NM_000224 | Homo sapiens keratin 18 (KRT18), transcript variant 1, mRNA. | 42 |
| NM_006819 | Homo sapiens stress-induced-phosphoprotein 1 (Hsp70/Hsp90-organizing protein) | 43 |
| | (STIP1), mRNA. | |
| XM_379877 | Homo sapiens similar to Nuclear envelope pore membrane protein POM 121 (Pore | 44 |
| | membrane protein of 121 kDa) (P145) (LOC402556), mRNA. | |
| NM_001436 | Homo sapiens fibrillarin (FBL), mRNA. | 45 |
| NM_004247 | Homo sapiens U5 snRNP-specific protein, 116 kD (U5-116 KD), mRNA. | 46 |
| NM_000967 | Homo sapiens ribosomal protein L3 (RPL3), mRNA. | 47 |
| NM_199413 | Homo sapiens protein regulator of cytokinesis 1 (PRC1), transcript variant 2, mRNA. | 48 |
| NM_032044 | Homo sapiens regenerating islet-derived family, member 4 (REG4), mRNA. | 49 |
| NM_013403 | Homo sapiens striatin, calmodulin binding protein 4 (STRN4), mRNA. | 50 |
| NM_012469 | Homo sapiens chromosome 20 open reading frame 14 (C20orf14), mRNA. | 51 |
| NM_003169 | Homo sapiens suppressor of Ty 5 homolog (S. cerevisiae) (SUPT5H), mRNA. | 52 |
| NM_006470 | Homo sapiens tripartite motif-containing 16 (TRIM16), mRNA. | 53 |
| NM_021991 | Homo sapiens junction plakoglobin (JUP), transcript variant 2, mRNA. | 54 |
| XM_290506 | Homo sapiens splicing factor 3b, subunit 2, 145 kDa (SF3B2), mRNA. | 55 |
| NM_153280 | Homo sapiens ubiquitin-activating enzyme E1 (A1S9T and BN75 temperature sensitivity | 56 |
| | complementing) (UBE1), transcript variant 2, mRNA. | |
| NM_080686 | Homo sapiens HLA-B associated transcript 2 (BAT2), transcript variant 1, mRNA. | 57 |
| NM_000289 | Homo sapiens phosphofructokinase, muscle (PFKM), mRNA. | 58 |
| NM_003875 | Homo sapiens guanine monphosphate synthetase (GMPS), mRNA. | 59 |
| NM_024658 | Homo sapiens importin 4 (IPO4), mRNA. | 60 |
| NM_003074 | Homo sapiens SWI/SNF related, matrix associated, actin dependent regulator of | 61 |
| | chromatin, subfamily c, member 1 (SMARCC1), mRNA. | |
| NM_152298 | Homo sapiens nuclear autoantigenic sperm protein (histone-binding) (NASP), transcript | 62 |
| | variant 3, mRNA. | |
| NM_007126 | Homo sapiens valosin-containing protein (VCP), mRNA. | 63 |
| NM_139215 | Homo sapiens TAF15 RNA polymerase II, TATA box binding protein (TBP)-associated | 64 |
| | factor, 68 kDa (TAF15), transcript variant 1, mRNA. | |
| NM_147200 | Homo sapiens chromosome 6 open reading frame 4 (C6orf4), transcript variant 1, mRNA. | 65 |
| XM_290345 | Homo sapiens similar to eukaryotic translation initiation factor 3, subunit 5 epsilon, | 66 |
| | 47 kDa; eukaryotic translation initiation factor 3, subunit 5 (epsilon, 47 kD); | |
| | eIF3-epsilon (LOC339799), mRNA. | |
| XM_377464 | Homo sapiens suppressor of Ty 6 homolog (S. cerevisiae) (SUPT6H), mRNA. | 67 |
| NM_021873 | Homo sapiens cell division cycle 25B (CDC25B), transcript variant 3, mRNA. | 68 |
| NM_006429 | Homo sapiens chaperonin containing TCP1, subunit 7 (eta) (CCT7), mRNA. | 69 |
| NM_015292 | Homo sapiens likely ortholog of mouse membrane bound C2 domain containing protein | 70 |
| | (MBC2), mRNA. | |
| NM_005956 | Homo sapiens methylenetetrahydrofolate dehydrogenase (NADP + dependent), | 71 |
| | methenyltetrahydrofolate cyclohydrolase, formyltetrahydrofolate synthetase (MTHFD1), | |
| | mRNA. | |
| NM_001940 | Homo sapiens dentatorubral-pallidoluysian atrophy (atrophin-1) (DRPLA), mRNA. | 72 |
| NM_000526 | Homo sapiens keratin 14 (epidermolysis bullosa simplex, Dowling-Meara, Koebner) | 73 |
| | (KRT14), mRNA. | |
| NM_001747 | Homo sapiens capping protein (actin filament), gelsolin-like (CAPG), mRNA. | 74 |
| NM_024311 | Homo sapiens hypothetical protein ET (ET), mRNA. | 75 |
| NM_003938 | Homo sapiens adaptor-related protein complex 3, delta 1 subunit (AP3D1), mRNA. | 76 |
| NM_005336 | Homo sapiens high density lipoprotein binding protein (vigilin) (HDLBP), mRNA. | 77 |
| NM_003751 | Homo sapiens eukaryotic translation initiation factor 3, subunit 9 eta, 116 kDa | 78 |
| | (EIF3S9), transcript variant 1, mRNA. | |
| NM_006839 | Homo sapiens inner membrane protein, mitochondrial (mitofilin) (IMMT), mRNA. | 79 |
| NM_000937 | Homo sapiens polymerase (RNA) II (DNA directed) polypeptide A, 220 kDa (POLR2A), | 80 |
| | mRNA. | |
| NM_012112 | Homo sapiens TPX2, microtubule-associated protein homolog (Xenopus laevis) (TPX2), | 81 |
| | mRNA. | |
| NM_006739 | Homo sapiens MCM5 minichromosome maintenance deficient 5, cell division cycle 46 | 82 |
| | (S. cerevisiae) (MCM5), mRNA. | |
| NM_005916 | Homo sapiens MCM7 minichromosome maintenance deficient 7 (S. cerevisiae) (MCM7), | 83 |
| | transcript variant 1, mRNA. | |
| NM_138421 | Homo sapiens hypothetical protein BC012010 (LOC113174), mRNA. | 84 |
| NM_001379 | Homo sapiens DNA (cytosine-5-)-methyltransferase 1 (DNMT1), mRNA. | 85 |
| NM_006289 | Homo sapiens talin 1 (TLN 1), mRNA. | 86 |
| NM_004939 | Homo sapiens DEAD (Asp-Glu-Ala-Asp) box polypeptide 1 (DDX1), mRNA. | 87 |
| NM_001357 | Homo sapiens DEAH (Asp-Glu-Ala-His) box polypeptide 9 (DHX9), transcript variant 1, | 88 |
| | mRNA. | |
| NM_000422 | Homo sapiens keratin 17 (KRT17), mRNA. | 89 |
| NM_002417 | Homo sapiens antigen identified by monoclonal antibody Ki-67 (MKI67), mRNA. | 90 |
| NM_005968 | Homo sapiens heterogeneous nuclear ribonucleoprotein M (HNRPM), transcript variant 1, | 91 |
| | mRNA. | |
| NM_003754 | Homo sapiens eukaryotic translation initiation factor 3, subunit 5 epsilon, 47 kDa | 92 |
| | (EIF3S5), mRNA. | |
| NM_032789 | Formerly XM_378197. Homo sapiens poly (ADP-ribose) polymerase family, | 93 |
| | member 10 (PARP10) | |
| NM_002473 | Homo sapiens myosin, heavy polypeptide 9, non-muscle (MYH9), mRNA. | 94 |
| NM_002972 | Homo sapiens SET binding factor 1 (SBF1), transcript variant 1, mRNA. | 95 |
| NM_000546 | Homo sapiens tumor protein p53 (Li-Fraumeni syndrome) (TP53), mRNA. | 96 |
| NM_001102 | Homo sapiens actinin, alpha 1 (ACTN1), mRNA. | 97 |
| XM_374522 | Homo sapiens similar to Filamin C (Gamma-filamin) (Filamin 2) (Protein FLNc) | 98 |
| | (Actin-binding like protein) (ABP-L) (ABP-280-like protein) (LOC392787), mRNA. | |
| NM_001458 | Homo sapiens filamin C, gamma (actin binding protein 280) (FLNC), mRNA. | 99 |
| NM_172020 | Homo sapiens POM121 membrane glycoprotein (rat) (POM121), mRNA. | 100 |
| NM_012416 | Homo sapiens RAN binding protein 6 (RANBP6), mRNA. Formerly XM_039701. | 101 |
| NM_000701 | Homo sapiens ATPase, Na+/K+ transporting, alpha 1 polypeptide (ATP1A1), mRNA. | 102 |
| NM_002298 | Homo sapiens lymphocyte cytosolic protein 1 (L-plastin) (LCP1), mRNA. | 103 |
| NM_015179 | Homo sapiens KIAA0690 (KIAA0690), mRNA. | 104 |
| NM_002967 | Homo sapiens scaffold attachment factor B (SAFB), mRNA. | 105 |
| NM_018454 | Homo sapiens nucleolar and spindle associated protein 1 (NUSAP1), mRNA. | 106 |
| NM_006796 | Homo sapiens AFG3 ATPase family gene 3-like 2 (yeast) (AFG3L2), nuclear gene encoding | 107 |
| | mitochondrial protein, mRNA. | |
| NM_005558 | Homo sapiens ladinin 1 (LAD1), mRNA. | 108 |
| NM_006812 | Homo sapiens amplified in osteosarcoma (OS-9), mRNA. | 109 |
| NM_003400 | Homo sapiens exportin 1 (CRM1 homolog, yeast) (XPO1), mRNA. | 110 |
| NM_001034 | Homo sapiens ribonucleotide reductase M2 polypeptide (RRM2), mRNA. | 111 |
| NM_004728 | Homo sapiens DEAD (Asp-Glu-Ala-Asp) box polypeptide 21 (DDX21), mRNA. | 112 |
| NM_178313 | Homo sapiens spectrin, beta, non-erythrocytic 1 (SPTBN1), transcript variant 2, mRNA. | 113 |
| NM_145714 | Homo sapiens ataxin 2 related protein (A2LP), transcript variant B, mRNA. | 114 |
| XM_085722 | Homo sapiens similar to Tripartite motif protein 16 (Estrogen-responsive B box | 115 |
| | protein) (LOC147166), mRNA. | |
| NM_001798 | Homo sapiens cyclin-dependent kinase 2 (CDK2), transcript variant 1, mRNA. | 116 |
| NM_181054 | Homo sapiens hypoxia-inducible factor 1, alpha subunit (basic helix-loop-helix | 117 |
| | transcription factor) (HIF1A), transcript variant 2, mRNA. | |
| NM_001916 | Homo sapiens cytochrome c-1 (CYC1), mRNA. | 118 |
| NM_002668 | Homo sapiens proteolipid protein 2 (colonic epithelium-enriched) (PLP2), mRNA. | 119 |
| NM_052963 | Homo sapiens mitochondrial topoisomerase I (TOP1MT), nuclear gene encoding | 120 |
| | mitochondrial protein, mRNA. | |
| NM_031243 | Homo sapiens heterogeneous nuclear ribonucleoprotein A2/B1 (HNRPA2B1), transcript | 121 |
| | variant 81, mRNA. | |
| NM_002638 | Homo sapiens protease inhibitor 3, skin-derived (SKALP) (PI3), mRNA. | 122 |
| NM_003146 | Homo sapiens structure specific recognition protein 1 (SSRP1), mRNA. | 123 |
| NM_005564 | Homo sapiens lipocalin 2 (oncogene 24p3) (LCN2), mRNA. | 124 |
| NM_016292 | Homo sapiens heat shock protein 75 (TRAP1), mRNA. | 125 |
| NM_018193 | Homo sapiens KIAA1794 (KIAA1794), mRNA. Formerly XM_375253. | 126 |
| NM_003815 | Homo sapiens a disintegrin and metalloproteinase domain 15 (metargidin) (ADAM15), | 127 |
| | mRNA. | |
| NM_001363 | Homo sapiens dyskeratosis congenita 1, dyskerin (DKC1), mRNA. | 128 |
| NM_032271 | Homo sapiens ring finger and WD repeat domain 1 (RFWD1), mRNA. | 129 |
| NM_000314 | Homo sapiens phosphatase and tensin homolog (mutated in multiple advanced cancers 1) | 130 |
| | (PTEN), mRNA. | |
| NM_198830 | Homo sapiens ATP citrate lyase (ACLY), transcript variant 2, mRNA. | 131 |
| NM_032999 | Homo sapiens general transcription factor II, i (GTF2I), transcript variant 1, mRNA. | 132 |
| NM_015935 | Homo sapiens CGI-01 protein (CGI-01), transcript variant 1, mRNA. | 133 |
| NM_134447 | Homo sapiens chromosome 19 open reading frame 2 (C19orf2), transcript variant 2, mRNA. | 134 |
| NM_006928 | Homo sapiens silver homolog (mouse) (SILV), mRNA. | 135 |
| NM_014390 | Homo sapiens staphylococcal nuclease domain containing 1 (SND1), mRNA. | 136 |
| NM_020117 | Homo sapiens leucyl-tRNA synthetase (LARS), mRNA. | 137 |
| NM_001619 | Homo sapiens adrenergic, beta, receptor kinase 1 (ADRBK1), mRNA. | 138 |
| NM_022820 | Homo sapiens cytochrome P450, family 3, subfamily A, polypeptide 43 (CYP3A43), | 139 |
| | transcript variant 1, mRNA. | |
| NM_005526 | Homo sapiens heat shock transcription factor 1 (HSF1), mRNA. | 140 |
| NM_033500 | Homo sapiens hexokinase 1 (HK1), nuclear gene encoding mitochondrial protein, | 141 |
| | transcript variant 5, mRNA. | |
| NM_001903 | Homo sapiens catenin (cadherin-associated protein), alpha 1, 102 kDa (CTNNA1), mRNA. | 142 |
| NM_004844 | Homo sapiens SH3-domain binding protein 5 (BTK-associated) (SH3BP5), mRNA. | 143 |
| NM_006372 | Homo sapiens synaptotagmin binding, cytoplasmic RNA interacting protein (SYNCRIP), | 144 |
| | mRNA. | |
| NM_022743 | Homo sapiens SET and MYND domain containing 3 (SMYD3), mRNA. | 145 |
| NM_007355 | Homo sapiens heat shock 90 kDa protein 1, beta (HSPCB), mRNA. | 146 |
| NM_012426 | Homo sapiens splicing factor 3b, subunit 3, 130 kDa (SF3B3), mRNA. | 147 |
| NM_000088 | Homo sapiens collagen, type I, alpha 1 (COL1A1), mRNA. | 148 |
| NM_182926 | Homo sapiens kinectin 1 (kinesin receptor) (KTN1), mRNA. | 149 |
| NM_004563 | Homo sapiens phosphoenolpyruvate carboxykinase 2 (mitochondrial) (PCK2), mRNA. | 150 |
| NM_014612 | Homo sapiens chromosome 9 open reading frame 10 (C9orf10), mRNA. | 151 |
| NM_004446 | Homo sapiens glutamyl-prolyl-tRNA synthetase (EPRS), mRNA. | 152 |
| XM_168585 | Homo sapiens similar to mucin 11 (LOC219612), mRNA. | 153 |
| NM_014173 | Homo sapiens HSPC142 protein (HSPC142), mRNA. | 154 |
| NM_144596 | Homo sapiens tetratricopeptide repeat domain 8 (TTC8), transcript variant 3, mRNA. | 155 |
| NM_138925 | Homo sapiens SON DNA binding protein (SON), transcript variant a, mRNA. | 156 |
| NM_174889 | Homo sapiens Myc-induced mitochondria protein (mimitin), mRNA | 157 |
| NM_006452 | Homo sapiens phosphoribosylaminoimidazole carboxylase, phosphoribosylaminoimidazole | 158 |
| | succinocarboxamide synthetase (PAICS), mRNA. | |
| NM_004689 | Homo sapiens metastasis associated 1 (MTA1), mRNA. | 159 |
| NM_015315 | Homo sapiens likely ortholog of mouse Ia related protein (LARP), mRNA. | 160 |
| XM_379904 | Homo sapiens similar to mucin 11 (LOC402575), mRNA. | 161 |
| NM_014753 | Homo sapiens BMS1-Iike, ribosome assembly protein (yeast) (BMS1L), mRNA. | 162 |
| NM_198309 | Homo sapiens tetratricopeptide repeat domain 8 (TTC8), transcript variant 1, mRNA. | 163 |
| NM_002810 | Homo sapiens proteasome (prosome, macropain) 26S subunit, non-ATPase, 4 (PSMD4), | 164 |
| | transcript variant 1, mRNA. | |
| NM_002388 | Homo sapiens MCM3 minichromosome maintenance deficient 3 (S. cerevisiae) (MCM3), | 165 |
| | mRNA. | |
| NM_014938 | Homo sapiens Mix interactor (MONDOA), mRNA. | 166 |
| NM_018031 | Homo sapiens WD repeat domain 6 (WDR6), mRNA. | 167 |
| NM_023007 | Homo sapiens jumonji domain containing 4 (JMJD4), mRNA | 168 |
| NM_002362 | Homo sapiens melanoma antigen, family A, 4 (MAGEA4), mRNA. | 169 |
| NM_006088 | Homo sapiens tubulin, beta, 2 (TUBB2), mRNA. | 170 |
| NM_002014 | Homo sapiens FK506 binding protein 4, 59 kDa (FKBP4), mRNA. | 171 |
| NM_003823 | Homo sapiens tumor necrosis factor receptor superfamily, member 6b, decoy (TNFRSF6B), | 172 |
| | transcript variant M68E, mRNA. | |
| NM_030877 | Homo sapiens catenin, beta like 1 (CTNNBL1), mRNA. | 173 |
| NM_003752 | Homo sapiens eukaryotic translation initiation factor 3, subunit 8, 110 kDa (EIF3S8), | 174 |
| | mRNA. | |
| NM_001456 | Homo sapiens filamin A, alpha (actin binding protein 280) (FLNA), mRNA. | 175 |
| NM_006286 | Homo sapiens transcription factor Dp-2 (E2F dimerization partner 2) (TFDP2), mRNA. | 176 |
| NM_145685 | Homo sapiens BRF1 homolog, subunit of RNA polymerase III transcription initiation | 177 |
| | factor IIIB (S. cerevisiae) (BRF1), transcript variant 3, mRNA. | |
| NM_003103 | Homo sapiens SON DNA binding protein (SON), transcript variant g, mRNA. | 178 |
| NM_002265 | Homo sapiens karyopherin (importin) beta 1 (KPNB1), mRNA. | 179 |
| NM_005915 | Homo sapiens MCM6 minichromosome maintenance deficient 6 (MIS5 homolog, S. pombe) | 180 |
| | (S. cerevisiae) (MCM6), mRNA. | |
| NM_006295 | Homo sapiens valyl-tRNA synthetase 2 (VARS2), mRNA. | 181 |
| NM_145810 | Homo sapiens cell division cycle associated 7 (CDCA7), transcript variant 2, mRNA. | 182 |
| NM_015456 | Homo sapiens cofactor of BRCA1 (COBRA1), mRNA. | 183 |

* All sequences are human.

The present invention encompasses methods of diagnosing whether a subject has, or is at risk for developing, a cancer, comprising determining the nucleotide sequence or the gene structure of one cancer gene in a test sample from the subject and comparing the nucleotide sequence or the gene structure of one cancer gene in a control sample. As used herein, a “subject” can be any mammal that has, or is suspected of having, a cancer. In a preferred embodiment, the subject is a human who has, or is suspected of having, a cancer.

In one embodiment, the at least one cancer gene assayed in the test sample is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof. In a particular embodiment, the cancer gene product is NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014 or NM_{—}001417. In another embodiment, the cancer gene product is not NM_{—}000546 or NM_{—}000314. In an additional embodiment, the cancer gene product is not NM_{—}198525 or XM_{—}093644. In an additional embodiment, the cancer gene product is not XM_{—}060328, NM_{—}173358, NM_{—}021014, NM_{—}005635, XM_{—}377964, XM_{—}380028, NM_{—}005636, NM_{—}173357, NM_{—}021015, NM_{—}021923, NM_{—}175698, NM_{—}024841, NM_{—}015055, NM_{—}016249, NM_{—}006771, XM_{—}377527, NM_{—}007109, NM_{—}020994, NM_{—}174961, NM_{—}172347, XM_{—}376672, XM_{—}374650, XM_{—}087062, NM_{—}001809, NM_{—}080746, XM_{—}372773, XM_{—}210557, NM_{—}175711, XM_{—}371019, XM_{—}371151, XM_{—}373345, NM_{—}001532, NM_{—}001941, NM_{—}080626, XM_{—}085138, XM_{—}376327, NM_{—}000298, NM_{—}006941, XM_{—}168583, XM_{—}379905, NM_{—}181871, NM_{—}005504, NM_{—}016932, NM_{—}003147, NM_{—}147199, NM_{—}054031, NM_{—}052969, NM_{—}001002, NM_{—}053275, NM_{—}005110, NM_{—}004153, NM_{—}133373, XM_{—}373373, XM_{—}016713, XM_{—}370872, NM_{—}152341, XM_{—}291704, XM_{—}371781, XM_{—}209178, XM_{—}371758, XM_{—}373089, NM_{—}017515, NM_{—}015061, NM_{—}012427, NM_{—}006546, XM_{—}379625, XM_{—}208043, NM_{—}005225, NM_{—}004140, NM_{—}032756, XM_{—}062300, NM_{—}021830, NM_{—}001252, NM_{—}002478, XM_{—}070233, or NM_{—}000315. In a further embodiment, the hypermutated cancer gene product is not NM_{—}002277, NM_{—}198696, NM_{—}052848, NM_{—}025108, NM_{—}018957, NM_{—}052817, NM_{—}020880, NM_{—}177478, NM_{—}019020, NM_{—}007197, NM_{—}006987, NM_{—}033316, NM_{—}012216, or NM_{—}152426. In another embodiment, the cancer gene product is not NM_{—}002034, NM_{—}018706, NM_{—}025265, XM_{—}045581, XM_{—}379215, XM_{—}372315, XM_{—}065899, NM_{—}005929, NM_{—}021795, NM_{—}004995, XM_{—}377110, NM_{—}020385, XM_{—}373949, XM_{—}377797, XM_{—}372916, XM_{—}372966, NM_{—}002280, XM_{—}377904, NM_{—}005686, NM_{—}033035, NM_{—}005547, XM_{—}292596, XM_{—}370710, NM_{—}024671, XM_{—}370988, NM_{—}006385, XM_{—}060535, NM_{—}031211, NM_{—}199182, XM_{—}378858, NM_{—}139177, XM_{—}371658, NM_{—}003771, NM_{—}018000, NM_{—}198855, XM_{—}085578, NM_{—}006681, NM_{—}018131, NM_{—}014321, NM_{—}005634, NM_{—}032792, NM_{—}001973, NM_{—}003202, NM_{—}024762, NM_{—}002278, NM_{—}005224, or NM_{—}001878. In yet another embodiment, the cancer gene product is not NM_{—}024087, NM_{—}022337, NM_{—}015990, NM_{—}002391, NM_{—}138430, NM_{—}023010, NM_{—}006465, NM_{—}018129, NM_{—}013373, NM_{—}017424, XM_{—}292627, NM_{—}003356, NM_{—}004739, NM_{—}153212, NM_{—}145060, XM_{—}371407, NM_{—}002386, XM_{—}372816, NM_{—}080632, XM_{—}370687, NM_{—}001805, or NM_{—}024888.

The cancer can be any cancer that arises from organs, solid or soft tissues, blood marrow. Such cancers are typically associated with the formation and/or presence of tumor masses and can be carcinomas, sarcomas, lymphomas or leukemias. Specific examples of cancers to be diagnosed by the methods of the invention include, but are not limited to, colon cancer, rectal cancer, stomach (gastric) cancer, pancreatic cancer, breast cancer, lung cancer, prostate cancer, bronchial cancer, testicular cancer, ovarian cancer, uterine cancer, penile cancer, melanoma and other skin cancers, liver cancer, esophageal cancer, cancers of the oral cavity and pharynx (e.g., tongue cancer, mouth cancer), cancers of the digestive system (e.g., intestinal cancer, gall bladder cancer), bone and joint cancers, cancers of the endocrine system (e.g., thyroid cancer), brain cancer, eye cancer, cancers of the urinary system (e.g., kidney cancer, urinary bladder cancer), Hodgkin disease and non-Hodgkin lymphoma

The nucleotide sequence or gene structure of at least one cancer gene can be measured in a biological sample (e.g., cells, tissues) obtained from the subject. For example, a tissue sample (e.g., from a tumor) can be removed from a subject suspected of having a cancer by conventional biopsy techniques. In another embodiment, a blood sample can be removed from the subject, and blood cells (e.g., white blood cells) can be isolated for DNA extraction by standard techniques. The blood or tissue sample is preferably obtained from the subject prior to initiation of radiotherapy, chemotherapy or other therapeutic treatment. A corresponding control tissue or blood sample can be obtained from unaffected tissues of the subject, from a normal human individual or population of normal individuals, or from cultured cells corresponding to the majority of cells in the subject's sample. The control tissue or blood sample is then processed along with the sample from the subject, so that the sequence or gene structure of cancer gene product produced from a given cancer gene in cells from the subject's sample can be compared to the corresponding cancer gene product from cells of the control sample.

An alteration (e.g., a mutation) in the gene structure of a cancer gene product in the sample obtained from the subject, relative to the level of a corresponding cancer gene product in a control sample, is indicative of the presence of a cancer in the subject. In one embodiment, the structure of the at least one cancer gene product in the test sample is mutated corresponding to the cancer gene product in the control sample (i.e., nucleotide sequence of the cancer gene product is “mutated”).

The nucleotide sequence or genetic structure of a cancer gene product in a sample can be measured using any technique that is suitable for detecting DNA mutations in a biological sample. Suitable techniques (e.g., Southern blotting, DNA sequencing, dHPLC, RT-PCR, CGH, array-CGH, in situ hybridization) for identifying DNA mutations or alterations in a biological sample (e.g., cells, tissues) are well known to those of skill in the art. In a particular embodiment, the nucleotide sequence of at least one cancer gene product is detected using DNA sequencing. For example, total cellular DNA can be purified from cells by homogenization in the presence of nucleic acid extraction buffer, followed by centrifugation. Nucleic acids are precipitated, and DNA is recovered. The cancer gene DNA is isolated by PCR amplification with cancer gene specific primers. Suitable methods for designing and synthesizing cancer gene specific primers are well known to those of skill in the art. See, for example, Molecular Cloning: A Laboratory Manual, J. Sambrook et al., eds., 3rd edition, Cold Spring Harbor Laboratory Press, 1989, the entire disclosure of which is incorporated by reference.

For example, the genomic DNA can be labeled with, e.g., a radionuclide, such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like.

DNA can be labeled to high specific activity by either the nick translation method of Rigby et al. (1977) J. Mol. Biol. 113:237-251 or by the random priming method of Fienberg et al. (1983), Anal. Biochem. 132:6-13, the entire disclosures of which are incorporated herein by reference. The latter is the method of choice for synthesizing ³²P_labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 108 cpm/microgram. Autoradiographic detection of hybridization can then be performed by exposing hybridized filters to photographic film. Densitometric scanning of the photographic films exposed by the hybridized filters provides an accurate measurement of array-CGH hybridizations. Using another approach, array-CGH hybridizations can be quantified by computerized imaging systems, such as the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to southern blotting, identifying genetic lesions can be accomplished using the technique of in situ hybridization. This technique requires fewer cells than the Southern blotting and array-CGH techniques, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given cancer gene product can be produced from the nucleic acid sequences provided in Table 1a and Table 1b, and include, but are not limited to, probes having about 90%, 95%, 98%, or 99% complementarity to a cancer gene product of interest, as described above.

In some instances, it may be desirable to simultaneously determine the genetic structure or nucleotide sequence of a plurality of different cancer gene products in a sample. In other instances, it may be desirable to determine the genetic structure or nucleotide sequence of all known cancer genes correlated with a cancer. Assessing cancer-specific genomic structures or nucleotide sequences for hundreds of cancer genes or gene products is time consuming and requires a large amount of total DNA or RNA (e.g., at least 20 μg for each Southern blot) and autoradiographic techniques that require radioactive isotopes.

To overcome these limitations, an oligonucleotide library, in microchip format (i.e., a microarray), may be constructed containing a set of oligonucleotide (e.g., oligodeoxynucleotides) probes that are specific for a set of cancer genes. Using such a microarray in an array-CGH assay, the genomic structure and nucleotide sequence of multiple cancer genes in a biological sample can be determined by laneling the DNA to generate a set of target oligodeoxynucleotides, and hybridizing them to probe the oligonucleotides on the microarray to generate a hybridization, or array-CGH, profile. The hybridization profile of the test sample can be compared to that of a control sample to determine which cancer genes have an altered genomic structure in cancer cells. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “cancer gene-specific probe oligonucleotide” or “probe oligonucleotide specific for a cancer gene” is meant a probe oligonucleotide that has a sequence selected to hybridize to a specific cancer gene gene product, or to a reverse transcript of the specific cancer gene gene product.

An “array-CGH profile” or “hybridization profile” of a particular sample is essentially a fingerprint of the state of the sample; while two states may have any particular gene similarly structured, the evaluation of a number of genes simultaneously allows the generation of a gene structure or sequence profile that is unique to the state of the cell. That is, normal tissue may be distinguished from cancerous (e.g. tumor) tissue, and within cancerous tissue, different prognosis states (for example, good or poor long term survival prospects) may be determined. By comparing genomic structure or nucleotide sequence profiles of cancer tissue in different states, information regarding which genes are important (including mutated, deleted or amplified genes) in each of these states is obtained. The identification of sequences that are altered in tumor tissues, resulting in different prognostic outcomes, allows the use of this information in a number of ways. For example, a particular treatment regime may be evaluated (e.g., to determine whether a chemotherapeutic drug acts to improve the long-term prognosis in a particular patient). Similarly, diagnosis may be done or confirmed by comparing patient samples with known genomic structure or nucleotide sequence profiles. Furthermore, these genomic structure or nucleotide sequence profiles (or individual cancer genes) allow screening of drug candidates that convert a tumor poor prognosis profile to a better prognosis profile.

Accordingly, the invention provides methods of diagnosing whether a subject has, or is at risk for developing, a cancer, comprising labeling DNA or RNA from a test sample obtained from the subject to provide a set of target oligodeoxynucleotides, hybridizing the target oligodeoxynucleotides to a microarray comprising cancer gene-specific probe oligonucleotides to provide a hybridization profile for the test sample, and comparing the test sample hybridization profile to a hybridization profile generated from a control sample, wherein an alteration in the signal of at least one cancer gene is indicative of the subject either having, or being at risk for developing, a cancer. In one embodiment, the microarray comprises cancer gene-specific probe oligonucleotides for a substantial portion of all described human hypermutated cancer genes. In a particular embodiment, the microarray comprises cancer gene-specific probe oligonucleotides for one or more hypermutated cancer genes selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

The microarray can be prepared from gene-specific oligonucleotide probes generated from hypermutated cancer genes sequences. The array may contain two or more different oligonucleotide probes for each hypermutated cancer genes, designed from different gene regions. The array may also contain for each oligonucleotide probe the 4 different nucleotide variations. This allows hybridization control and determination of the nucleotide sequence. One or more appropriate controls for non-specific hybridization may also be included on the microchip. For this purpose, sequences are selected based upon the absence of any homology with any human DNA or RNA sequence.

The microarray may be fabricated using techniques known in the art. For example, probe oligonucleotides of an appropriate length, e.g., 60 nucleotides, are 5′-amine modified at position C6 and printed using commercially available microarray systems, e.g., the Biorobotics Arrayer and Amersham CodeLink™ activated slides. Labeled DNA oligomer corresponding to the target DNA is prepared by copying and labeling the genomic DNA with random primer. The labeled target DNAs thus prepared are then hybridized to the microarray chip under hybridizing conditions, e.g., 6×SSPE/30% formamide at 42° C. for 18 hours, followed by washing in 0.5×SSC and 0.1% SDS at 37° C. for 40 minutes. At positions on the array where the immobilized probe DNA recognizes a complementary target DNA in the sample, hybridization occurs. The labeled target DNA marks the exact position on the array where binding occurs, allowing automatic detection and quantification. The output consists of a list of hybridization events, indicating the relative abundance of specific DNA sequences, and therefore the relative abundance of the corresponding complementary hypermutated cancer gene, in the patient sample. According to one embodiment, the labeled DNA oligomer is a Cy3-labeled DNA. The microarray is then scanned utilizing conventional scanning methods. Image intensities of each spot on the array are proportional to the abundance of the corresponding hypermutated cancer gene in the patient sample.

The use of the array has several advantages for hypermutated cancer gene copy structural determination. First, the global structural genetic status of several hundred genes can be identified in the same sample at one time point. Second, through careful design of the oligonucleotide probes internal gene rearrangements can be identified. Third, in comparison with Southern blot analysis, the chip requires a small amount of DNA, and provides reproducible results using 2 μg of genomic DNA. Since the assayed regions for each cancer gene are only the exons (coding or non-coding) RNA can be used in place of genomic DNA. The relatively limited number of hypermutated cancer gene (less than 1% of the total human genes) allows the construction of a high detail microarray, with distinct oligonucleotide probes for each frequent genetic alteration. Such a tool would allow for analysis of genetic alterations for many different cancer types under various conditions.

In addition to use for structural genetic assays of specific cancer genes, a microchip containing cancer genes-specific probe oligonucleotides corresponding to a substantial portion of the cancer hypermutome, preferably the entire hypermutome, may be employed to carry out cancer genes gene structural profiling, for analysis of cancer genes mutation patterns. Distinct cancer genes signatures can be associated with established disease markers, or directly with a disease state.

According to the mutation profiling methods described herein, DNA or total RNA from a sample from a subject suspected of having a cancer (e.g., a solid cancer) is processed to provide a set of labeled target oligodeoxynucleotides complementary to the DNA or RNA in the sample. The target oligodeoxynucleotides are then hybridized to a microarray comprising hypermutated cancer gene-specific probe oligonucleotides to provide a hybridization profile for the sample. The result is a hybridization profile for the sample representing the mutation pattern of hypermutated cancer gene in the sample. The hybridization profile comprises the signal from the binding of the target oligodeoxynucleotides from the sample to the hypermutated cancer gene-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a normal, i.e., noncancerous, control sample. An alteration in the signal is indicative of the presence of, or propensity to develop, cancer in the subject. Other techniques for measuring hypermutated cancer gene mutations are also within the skill in the art, and include various techniques for determining nucleotide sequences.

The invention also provides methods of determining the prognosis of a subject with a cancer, comprising assaying the mutations of at least hypermutated cancer gene product, which is associated with a particular prognosis in a cancer (e.g., a good or positive prognosis, a poor or adverse prognosis), in a test sample from the subject. According to these methods, an alteration in the nucleotide sequence or genomic structure of a hypermutated cancer gene product that is associated with a particular prognosis, in the test sample, as compared to the nucleotide sequence or genomic structure of a corresponding hypermutated cancer gene product in a control sample, is indicative of the subject having a cancer with a particular prognosis. In one embodiment, the hypermutated cancer gene gene product is associated with an adverse (i.e., poor) prognosis. Examples of an adverse prognosis include, but are not limited to, low survival rate and rapid disease progression. In certain embodiments, the genomic structure or nucleotide sequence of the at least one hypermutated cancer gene gene product is assayed by labeling the genomic DNA or by reverse transcribing RNA from a test sample obtained from the subject to provide a set of target oligodeoxynucleotides, hybridizing the target oligodeoxynucleotides to a microarray that comprises hypermutated cancer gene-specific probe oligonucleotides to provide a hybridization profile for the test sample, and comparing the test sample hybridization profile to a hybridization profile generated from a control sample.

Alterations in the nucleotide sequence or the genomic structure of one or more hypermutated cancer gene products in cells can result in the deregulation of the corresponding protein or in the production of an aberrant protein, which can lead to the formation of cancers. Therefore, altering the activity of the hypermutated cancer gene product (e.g., by decreasing the level of a aberrant hypermutated cancer gene product present in cancer cells, or by increasing the level of a wild type hypermutated cancer gene product that is lacking in cancer cells) may successfully treat the cancer.

Accordingly, the present invention encompasses methods of inhibiting tumorigenesis in subject who has, or is suspected of having, a cancer wherein at least one hypermutated cancer gene product is deregulated in the cancer cells of the subject. When the at least one isolated hypermutated cancer gene product is aberrant or lacking in the cancer cells, the method comprises administering an effective amount of the at least one isolated hypermutated cancer gene product, or an isolated variant or biologically-active fragment thereof, such that proliferation of cancer cells in the subject is inhibited. For example, when a hypermutated cancer gene product is down-regulated in a cancer cell in a subject, administering an effective amount of an isolated hypermutated cancer gene product to the subject can inhibit proliferation of the cancer cell. The isolated hypermutated cancer gene product that is administered to the subject can be identical to the endogenous wild-type hypermutated cancer gene product (e.g., a hypermutated cancer gene product shown in Table 1) that is down-regulated in the cancer cell or it can be a variant or biologically-active fragment thereof. As defined herein, a “variant” of a hypermutated cancer gene product refers to a hypermutated cancer gene that has less than 100% identity to a corresponding wild-type hypermutated cancer gene product and is capable of producing a wild type protein. Such variants includes species variants and variants that are the consequence of one or more mutations in a hypermutated cancer gene. In certain embodiments, the variant is at least about 70%, 75%, 80%, 85%, 90%, 95%, 98%, or 99% identical to a corresponding wild-type hypermutated cancer gene product. As defined herein, a “biologically-active fragment” of a hypermutated cancer gene product refers to an RNA or DNA fragment of a hypermutated cancer gene product that is capable of producing a wild-type or functional hypermutated cancer gene product An isolated hypermutated cancer gene product, or an isolated variant or biologically-active fragment thereof, can be administered to a subject in combination with one or more additional anti-cancer treatments. Suitable anti-cancer treatments include, but are not limited to, chemotherapy, radiation therapy and combinations thereof (e.g., chemoradiation).

When the at least one isolated hypermutated cancer gene product is up-regulated in the cancer cells, the method comprises administering to the subject an effective amount of at least one compound for inhibiting expression of the at least one hypermutated cancer gene product, referred to herein as hypermutated cancer gene expression-inhibition compounds, such that proliferation of cancer cells is inhibited. In a particular embodiment, the at least one hypermutated cancer gene expression-inhibition compound is specific for a hypermutated cancer gene product selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof. A hypermutated cancer gene expression-inhibiting compound can be administered to a subject in combination with one or more additional anti-cancer treatments. Suitable anti-cancer treatments include, but are not limited to, chemotherapy, radiation therapy and combinations thereof (e.g., chemoradiation).

In one embodiment, the isolated cancer gene assayed in the test sample is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof. In a particular embodiment, the cancer gene product is NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014 or NM_{—}001417. In another embodiment, the cancer gene product is not NM_{—}000546 or NM_{—}000314. In an additional embodiment, the cancer gene product is not NM_{—}198525 or XM_{—}093644. In an additional embodiment, the cancer gene product is not XM_{—}060328, NM_{—}173358, NM_{—}021014, NM_{—}005635, XM_{—}377964, XM_{—}380028, NM_{—}005636, NM_{—}173357, NM_{—}021015, NM_{—}021923, NM_{—}175698, NM_{—}024841, NM_{—}015055, NM_{—}016249, NM_{—}006771, XM_{—}377527, NM_{—}007109, NM_{—}020994, NM_{—}174961, NM_{—}172347, XM_{—}376672, XM_{—}374650, XM_{—}087062, NM_{—}001809, NM_{—}080746, XM_{—}372773, XM_{—}210557, NM_{—}175711, XM_{—}371019, XM_{—}371151, XM_{—}373345, NM_{—}001532, NM_{—}001941, NM_{—}080626, XM_{—}085138, XM_{—}376327, NM_{—}000298, NM_{—}006941, XM_{—}168583, XM_{—}379905, NM_{—}181871, NM_{—}005504, NM_{—}016932, NM_{—}003147, NM_{—}147199, NM_{—}054031, NM_{—}052969, NM_{—}001002, NM_{—}053275, NM_{—}005110, NM_{—}004153, NM_{—}133373, XM_{—}373373, XM_{—}016713, XM_{—}370872, NM_{—}152341, XM_{—}291704, XM_{—}371781, XM_{—}209178, XM_{—}371758, XM_{—}373089, NM_{—}017515, NM_{—}015061, NM_{—}012427, NM_{—}006546, XM_{—}379625, XM_{—}208043, NM_{—}005225, NM_{—}004140, NM_{—}032756, XM_{—}062300, NM_{—}021830, NM_{—}001252, NM_{—}002478, XM_{—}070233, or NM_{—}000315. In a further embodiment, the hypermutated cancer gene product is not NM_{—}002277, NM_{—}198696, NM_{—}052848, NM_{—}025108, NM_{—}018957, NM_{—}052817, NM_{—}020880, NM_{—}177478, NM_{—}019020, NM_{—}007197, NM_{—}006987, NM_{—}033316, NM_{—}012216, or NM_{—}152426. In another embodiment, the cancer gene product is not NM_{—}002034, NM_{—}018706, NM_{—}025265, XM_{—}045581, XM_{—}379215, XM_{—}372315, XM_{—}065899, NM_{—}005929, NM_{—}021795, NM_{—}004995, XM_{—}377110, NM_{—}020385, XM_{—}373949, XM_{—}377797, XM_{—}372916, XM_{—}372966, NM_{—}002280, XM_{—}377904, NM_{—}005686, NM_{—}033035, NM_{—}005547, XM_{—}292596, XM_{—}370710, NM_{—}024671, XM_{—}370988, NM_{—}006385, XM_{—}060535, NM_{—}031211, NM_{—}199182, XM_{—}378858, NM_{—}139177, XM_{—}371658, NM_{—}003771, NM_{—}018000, NM_{—}198855, XM_{—}085578, NM_{—}006681, NM_{—}018131, NM_{—}014321, NM_{—}005634, NM_{—}032792, NM_{—}001973, NM_{—}003202, NM_{—}024762, NM_{—}002278, NM_{—}005224, or NM_{—}001878. In yet another embodiment, the cancer gene product is not NM_{—}024087, NM_{—}022337, NM_{—}015990, NM_{—}002391, NM_{—}138430, NM_{—}023010, NM_{—}006465, NM_{—}018129, NM_{—}013373, NM_{—}017424, XM_{—}292627, NM_{—}003356, NM_{—}004739, NM_{—}153212, NM_{—}145060, XM_{—}371407, NM_{—}002386, XM_{—}372816, NM_{—}080632, XM_{—}370687, NM_{—}001805, or NM_{—}024888.

The terms “treat”, “treating” and “treatment”, as used herein, refer to ameliorating symptoms associated with a disease or condition, for example, a cancer, including preventing or delaying the onset of the disease symptoms, and/or lessening the severity or frequency of symptoms of the disease or condition. The terms “subject” and “individual” are defined herein to include animals, such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “effective amount” of an isolated hypermutated cancer gene product is an amount sufficient to inhibit proliferation of a cancer cell in a subject suffering from a cancer. One skilled in the art can readily determine an effective amount of a hypermutated cancer gene product to be administered to a given subject, by taking into account factors, such as the size and weight of the subject; the extent of disease penetration; the age, health and sex of the subject, the route of administration, and whether the administration is regional or systemic.

For example, an effective amount of an isolated hypermutated cancer gene product can be based on the approximate weight of a tumor mass to be treated. The approximate weight of a tumor mass can be determined by calculating the approximate volume of the mass, wherein one cubic centimeter of volume is roughly equivalent to one gram. An effective amount of the isolated hypermutated cancer gene product based on the weight of a tumor mass can be in the range of about 10-500 micrograms/gram of tumor mass. In certain embodiments, the tumor mass can be at least about 10 micrograms/gram of tumor mass, at least about 60 micrograms/gram of tumor mass or at least about 100 micrograms/gram of tumor mass.

An effective amount of an isolated hypermutated cancer gene product can also be based on the approximate or estimated body weight of a subject to be treated. Preferably, such effective amounts are administered parenterally or enterally, as described herein. For example, an effective amount of the isolated hypermutated cancer gene product is administered to a subject can range from about 5-3000 micrograms/kg of body weight, from about 700-1000 micrograms/kg of body weight, or greater than about 1000 micrograms/kg of body weight.

One skilled in the art can also readily determine an appropriate dosage regimen for the administration of an isolated hypermutated cancer gene product to a given subject. For example, a hypermutated cancer gene product can be administered to the subject once (e.g., as a single injection or deposition). Alternatively, a hypermutated cancer gene product can be administered once or twice daily to a subject for a period of from about three to about twenty-eight days, more particularly from about seven to about ten days. In a particular dosage regimen, a hypermutated cancer gene product is administered once a day for seven days. Where a dosage regimen comprises multiple administrations, it is understood that the effective amount of the hypermutated cancer gene product administered to the subject can comprise the total amount of gene product administered over the entire dosage regimen.

As used herein, an “isolated” hypermutated cancer gene product is one that is synthesized, or altered or removed from the natural state through human intervention. For example, a synthetic hypermutated cancer gene product, or a hypermutated cancer gene product partially or completely separated from the coexisting materials of its natural state, is considered to be “isolated.” An isolated hypermutated cancer gene product can exist in substantially-purified form, or can exist in a cell into which the hypermutated cancer gene product has been delivered. Thus, a hypermutated cancer gene product that is deliberately delivered to, or expressed in, a cell is considered an “isolated” hypermutated cancer gene product. A hypermutated cancer gene product produced inside a cell from a messenger RNA is also considered to be an “isolated” molecule. According to the invention, the isolated hypermutated cancer gene products described herein can be used for the manufacture of a medicament for treating a cancer in a subject (e.g., a human).

Isolated hypermutated cancer gene products can be obtained using a number of standard techniques. For example, the hypermutated cancer gene products can be chemically synthesized or recombinantly produced using methods known in the art. In one embodiment, hypermutated cancer gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the hypermutated cancer gene products can be expressed from recombinant circular or linear DNA plasmids using any suitable promoter. Suitable promoters for expressing RNA from a plasmid include, e.g., the U6 or Hi RNA pol III promoter sequences, or the cytomegalovirus promoters. Selection of other suitable promoters is within the skill in the art. The recombinant plasmids of the invention can also comprise inducible or regulatable promoters for expression of the hypermutated cancer gene products in cancer cells.

The hypermutated cancer gene products that are expressed from recombinant plasmids can be isolated from cultured cell expression systems by standard techniques. The hypermutated cancer gene products that are expressed from recombinant plasmids can also be delivered to, and expressed directly in, the cancer cells. The use of recombinant plasmids to deliver the hypermutated cancer gene products to cancer cells is discussed in more detail below.

The hypermutated cancer gene products can be expressed from a separate recombinant plasmid, or they can be expressed from the same recombinant plasmid. In one embodiment, the hypermutated cancer gene products are expressed as RNA precursor molecules from a single plasmid, and the precursor molecules are processed into the functional hypermutated cancer gene product by a suitable processing system, including, but not limited to, processing systems extant within a cancer cell. Other suitable processing systems include, e.g., the in vitro Drosophila cell lysate system (e.g., as described in U.S. Published Patent Application No. 2002/0086356 to Tuschl et al., the entire disclosure of which is incorporated herein by reference) and the E. coli RNAse III system (e.g., as described in U.S. Published Patent Application No. 2004/0014113 to Yang et al., the entire disclosure of which is incorporated herein by reference).

Selection of plasmids suitable for expressing the hypermutated cancer gene products, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al (2002), Nat. Biotechnol. 20:500-505; and Paul et al. (2002), Nat. Biotechnol. 20:505-508, the entire disclosures of which are incorporated herein by reference.

In one embodiment, a plasmid expressing the hypermutated cancer gene products comprises a sequence encoding a hypermutated cancer gene RNA under the control of the CMV intermediate-early promoter. As used herein, “under the control” of a promoter means that the nucleic acid sequences encoding the hypermutated cancer gene product are located 3′ of the promoter, so that the promoter can initiate transcription of the hypermutated cancer gene product coding sequences.

The hypermutated cancer gene products can also be expressed from recombinant viral vectors. It is contemplated that the hypermutated cancer gene products can be expressed from two separate recombinant viral vectors, or from the same viral vector. The RNA expressed from the recombinant viral vectors can either be isolated from cultured cell expression systems by standard techniques, or can be expressed directly in cancer cells. The use of recombinant viral vectors to deliver the hypermutated cancer gene products to cancer cells is discussed in more detail below.

The recombinant viral vectors of the invention comprise sequences encoding the hypermutated cancer gene products and any suitable promoter for expressing the RNA sequences. Suitable promoters include, but are not limited to, the U6 or H1 RNA pol III promoter sequences, or the cytomegalovirus promoters. Selection of other suitable promoters is within the skill in the art. The recombinant viral vectors of the invention can also comprise inducible or regulatable promoters for expression of the hypermutated cancer gene products in a cancer cell.

Any viral vector capable of accepting the coding sequences for the hypermutated cancer gene products can be used; for example, vectors derived from adenovirus (AV); adeno-associated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors that express different capsid protein serotypes are within the skill in the art; see, e.g., Rabinowitz, J. E., et al. (2002), J. Virol. 76:791-801, the entire disclosure of which is incorporated herein by reference.

Selection of recombinant viral vectors suitable for use in the invention, methods for inserting nucleic acid sequences for expressing RNA into the vector, methods of delivering the viral vector to the cells of interest, and recovery of the expressed RNA products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are incorporated herein by reference.

Particularly suitable viral vectors are those derived from AV and AAV. A suitable AV vector for expressing the hypermutated cancer gene products, a method for constructing the recombinant AV vector, and a method for delivering the vector into target cells, are described in Xia et al. (2002), Nat. Biotech. 20:1006-1010, the entire disclosure of which is incorporated herein by reference. Suitable AAV vectors for expressing the hypermutated cancer gene products, methods for constructing the recombinant AAV vector, and methods for delivering the vectors into target cells are described in Samulski et al. (1987), J. Virol. 61:3096-3101; Fisher et al. (1996), J. Virol, 70:520-532; Samulski et al. (1989), J. Virol. 63:3822-3826; U.S. Pat. No. 5,252,479; U.S. Pat. No. 5,139,941; International Patent Application No. WO 94/13788; and International Patent Application No. WO 93/24641, the entire disclosures of which are incorporated herein by reference. In one embodiment, the hypermutated cancer gene products are expressed from a single recombinant AAV vector comprising the CMV intermediate early promoter.

In a certain embodiment, a recombinant AAV viral vector of the invention comprises a nucleic acid sequence encoding a hypermutated cancer gene RNA in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the hypermutated cancer gene sequences from the vector, the polyT termination signals act to terminate transcription.

In other embodiments of the treatment methods of the invention, an effective amount of at least one compound that inhibits hypermutated cancer gene product activity can be administered to the subject. As used herein, “inhibiting hypermutated cancer gene product activity” means that the production of the active form of hypermutated cancer gene product after treatment is less than the amount produced prior to treatment. One skilled in the art can readily determine whether hypermutated cancer gene product activity has been inhibited in a cancer cell, using, for example, the techniques for determining cancer gene transcript or protein product level discussed above for the diagnostic method. Inhibition can occur at the level of gene expression (i.e., by inhibiting transcription of a cancer gene encoding the hypermutated cancer gene product).

As used herein, an “effective amount” of a compound that inhibits hypermutated cancer gene product activity is an amount sufficient to inhibit proliferation of a cancer cell in a subject suffering from a cancer (e.g., a solid cancer). One skilled in the art can readily determine an effective amount of a hypermutated cancer gene product activity-inhibition compound to be administered to a given subject, by taking into account factors, such as the size and weight of the subject; the extent of disease penetration; the age, health and sex of the subject, the route of administration, and whether the administration is regional or systemic.

For example, an effective amount of the expression-inhibition compound can be based on the approximate weight of a tumor mass to be treated, as described herein. An effective amount of a compound that inhibits hypermutated cancer gene product activity can also be based on the approximate or estimated body weight of a subject to be treated, as described herein. One skilled in the art can also readily determine an appropriate dosage regimen for administering a compound that inhibits hypermutated cancer gene product activity to a given subject.

Suitable compounds for inhibiting hypermutated cancer gene expression include double-stranded RNA (such as short- or small-interfering RNA or “siRNA”), antisense nucleic acids, and enzymatic RNA molecules, such as ribozymes. Each of these compounds can be targeted to a given hypermutated cancer gene product and interfere with the expression of (e.g., inhibit translation of, induce cleavage or destruction of) the target hypermutated cancer gene product.

For example, expression of a given hypermutated cancer gene can be inhibited by inducing RNA interference of the hypermutated cancer gene with an isolated double-stranded RNA (“dsRNA”) molecule which has at least 90%, for example at least 95%, at least 98%, at least 99%, or 100%, sequence homology with at least a portion of the hypermutated cancer gene product. In a particular embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence that is substantially identical to a nucleic acid sequence contained within the target hypermutated cancer gene product.

As used herein, a nucleic acid sequence in an siRNA which is “substantially identical” to a target sequence contained within the target mRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area.

The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA can also comprise a 3′ overhang. As used herein, a “3′ overhang” refers to at least one unpaired nucleotide extending from the 3′-end of a duplexed RNA strand. Thus, in certain embodiments, the siRNA comprises at least one 3′ overhang of from 1 to about 6 nucleotides (which includes ribonucleotides or deoxyribonucleotides) in length, from 1 to about 5 nucleotides in length, from 1 to about 4 nucleotides in length, or from about 2 to about 4 nucleotides in length. In a particular embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated hypermutated cancer gene products. Exemplary methods for producing and testing dsRNA or siRNA molecules are described in U.S. Published Patent Application No. 2002/0173478 to Gewirtz and in U.S. Published Patent Application No. 2004/0018176 to Reich et al., the entire disclosures of both of which are incorporated herein by reference.

Expression of a given hypermutated cancer gene can also be inhibited by an antisense nucleic acid. As used herein, an “antisense nucleic acid” refers to a nucleic acid molecule that binds to target RNA by means of RNA-RNA, RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, peptide nucleic acid (PNA)) that generally comprise a nucleic acid sequence complementary to a contiguous nucleic acid sequence in a hypermutated cancer gene product. The antisense nucleic acid can comprise a nucleic acid sequence that is 50-100% complementary, 75-100% complementary, or 95-100% complementary to a contiguous nucleic acid sequence in a hypermutated cancer gene product. Nucleic acid sequences for the hypermutated cancer gene products are provided in Table 1. Without wishing to be bound by any theory, it is believed that the antisense nucleic acids activate RNase H or another cellular nuclease that digests the hypermutated cancer gene product/antisense nucleic acid duplex.

Antisense nucleic acids can also contain modifications to the nucleic acid backbone or to the sugar and base moieties (or their equivalent) to enhance target specificity, nuclease resistance, delivery or other properties related to efficacy of the molecule. Such modifications include cholesterol moieties, duplex intercalators, such as acridine, or one or more nuclease-resistant groups.

Antisense nucleic acids can be produced chemically or biologically, or can be expressed from a recombinant plasmid or viral vector, as described above for the isolated hypermutated cancer gene products. Exemplary methods for producing and testing are within the skill in the art; see, e.g., Stein and Cheng (1993), Science 261:1004 and U.S. Pat. No. 5,849,902 to Woolf et al., the entire disclosures of which are incorporated herein by reference.

Expression of a given hypermutated cancer gene can also be inhibited by an enzymatic nucleic acid. As used herein, an “enzymatic nucleic acid” refers to a nucleic acid comprising a substrate binding region that has complementarity to a contiguous nucleic acid sequence of a hypermutated cancer gene product, and which is able to specifically cleave the hypermutated cancer gene product. The enzymatic nucleic acid substrate binding region can be, for example, 50-100% complementary, 75-100% complementary, or 95-100% complementary to a contiguous nucleic acid sequence in a hypermutated cancer gene product. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated hypermutated cancer gene products. Exemplary methods for producing and testing dsRNA or siRNA molecules are described in Werner and Uhlenbeck (1995), Nucl. Acids Res. 23:2092-96; Hammann et al. (1999), Antisense and Nucleic Acid Drug Dev. 9:25-31; and U.S. Pat. No. 4,987,071 to Cech et al, the entire disclosures of which are incorporated herein by reference.

Administration of at least one hypermutated cancer gene product, or at least one compound for inhibiting hypermutated cancer gene product activity, will inhibit the proliferation of cancer cells in a subject who has a cancer. As used herein, to “inhibit the proliferation of a cancer cell” means to kill the cell, or permanently or temporarily arrest or slow the growth of the cell. Inhibition of cancer cell proliferation can be inferred if the number of such cells in the subject remains constant or decreases after administration of the hypermutated cancer gene products or hypermutated cancer gene expression-inhibition compounds. An inhibition of cancer cell proliferation can also be inferred if the absolute number of such cells increases, but the rate of tumor growth decreases.

The number of cancer cells in the body of a subject can be determined by direct measurement, or by estimation from the size of primary or metastatic tumor masses. For example, the number of cancer cells in a subject can be measured by immunohistological methods, flow cytometry, or other techniques designed to detect characteristic surface markers of cancer cells.

The size of a tumor mass can be ascertained by direct visual observation, or by diagnostic imaging methods, such as X-ray, magnetic resonance imaging, ultrasound, and scintigraphy. Diagnostic imaging methods used to ascertain size of the tumor mass can be employed with or without contrast agents, as is known in the art. The size of a tumor mass can also be ascertained by physical means, such as palpation of the tissue mass or measurement of the tissue mass with a measuring instrument, such as a caliper.

The hypermutated cancer gene products or hypermutated cancer gene expression-inhibition compounds can be administered to a subject by any means suitable for delivering these compounds to cancer cells of the subject. For example, the hypermutated cancer gene products or hypermutated cancer gene product activity-inhibition compounds can be administered by methods suitable to transfect cells of the subject with these compounds, or with nucleic acids comprising sequences encoding these compounds. In one embodiment, the cells are transfected with a plasmid or viral vector comprising sequences encoding at least one hypermutated cancer gene product or hypermutated cancer gene expression-inhibition compound.

Transfection methods for eukaryotic cells are well known in the art, and include, e.g., direct injection of the nucleic acid into the nucleus or pronucleus of a cell; electroporation; liposome transfer or transfer mediated by lipophilic materials; receptor-mediated nucleic acid delivery, bioballistic or particle acceleration; calcium phosphate precipitation, and transfection mediated by viral vectors.

For example, cells can be transfected with a liposomal transfer compound, e.g., DOTAP (N-[1-(2,3-dioleoyloxy)propyl]-N,N,N-trimethyl-ammonium methylsulfate, Boehringer-Mannheim) or an equivalent, such as LIPOFECTIN. The amount of nucleic acid used is not critical to the practice of the invention; acceptable results may be achieved with 0.1-100 micrograms of nucleic acid/105 cells. For example, a ratio of about 0.5 micrograms of plasmid vector in 3 micrograms of DOTAP per 105 cells can be used.

A hypermutated cancer gene product or hypermutated cancer gene expression-inhibition compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Particularly suitable administration routes are injection, infusion and direct injection into the tumor.

In the present methods, a hypermutated cancer gene product or hypermutated cancer gene product expression-inhibition compound can be administered to the subject either as naked RNA, in combination with a delivery reagent, or as a nucleic acid (e.g., a recombinant plasmid or viral vector) comprising sequences that express the hypermutated cancer gene product or hypermutated cancer gene product expression-inhibition compound. Suitable delivery reagents include, e.g., the Mirus Transit TKO lipophilic reagent; lipofectin; lipofectamine; cellfectin; polycations (e.g., polylysine), and liposomes.

Recombinant plasmids and viral vectors comprising sequences that express the hypermutated cancer gene products or hypermutated cancer gene expression-inhibition compounds, and techniques for delivering such plasmids and vectors to cancer cells, are discussed herein and/or are well known in the art.

In a particular embodiment, liposomes are used to deliver a hypermutated cancer gene product or hypermutated cancer gene expression-inhibition compound (or nucleic acids comprising sequences encoding them) to a subject. Liposomes can also increase the blood half-life of the gene products or nucleic acids. Suitable liposomes for use in the invention can be formed from standard vesicle-forming lipids, which generally include neutral or negatively charged phospholipids and a sterol, such as cholesterol. The selection of lipids is generally guided by consideration of factors, such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are incorporated herein by reference. The liposomes for use in the present methods can comprise a ligand molecule that targets the liposome to cancer cells. Ligands that bind to receptors prevalent in cancer cells, such as monoclonal antibodies that bind to tumor cell antigens, are preferred. The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both an opsonization-inhibition moiety and a ligand.

Opsonization-inhibiting moieties for use in preparing the liposomes of the invention are typically large hydrophilic polymers that are bound to the liposome membrane. As used herein, an opsonization-inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is incorporated herein by reference.

Opsonization-inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers, such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization-inhibiting polymer can be a block copolymer of PEG and either a polyamino acid, polysaccharide, polyamidoamine, polyethyleneamine, or polynucleotide. The opsonization-inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or a derivative thereof. Liposomes modified with PEG or PEG-derivatives are sometimes called “PEGylated liposomes.”

The opsonization-inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example, solid tumors, will efficiently accumulate these liposomes; see Gabizon, et al. (1988), Proc. Natl. Acad. Sci., U.S.A., 18:6949-53. In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the hypermutated cancer gene products or hypermutated cancer gene expression-inhibition compounds (or nucleic acids comprising sequences encoding them) to tumor cells.

The hypermutated cancer gene products or hypermutated cancer gene expression-inhibition compounds can be formulated as pharmaceutical compositions, sometimes called “medicaments,” prior to administering them to a subject, according to techniques known in the art. Accordingly, the invention encompasses pharmaceutical compositions for treating a cancer. In one embodiment, the pharmaceutical composition comprises at least one isolated hypermutated cancer gene product, or an isolated variant or biologically-active fragment thereof, and a pharmaceutically-acceptable carrier. In a particular embodiment, the at least one hypermutated cancer gene product corresponds to a hypermutated cancer gene product that has a altered gene structure in cancer cells relative to suitable control celisin other embodiments, the pharmaceutical compositions of the invention comprise at least one hypermutated cancer gene product activity-inhibition compound In certain embodiments, the hypermutated cancer gene expression-inhibition compound is specific for one or more hypermutated cancer gene products selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

Pharmaceutical compositions of the present invention are characterized as being at least sterile and pyrogen-free. As used herein, “pharmaceutical compositions” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is incorporated herein by reference.

The present pharmaceutical compositions comprise at least one hypermutated cancer gene product or hypermutated cancer gene expression-inhibition compound (or at least one nucleic acid comprising sequences encoding them) (e.g., 0.1 to 90% by weight), or a physiologically-acceptable salt thereof, mixed with a pharmaceutically-acceptable carrier. In certain embodiments, the pharmaceutical compositions of the invention additionally comprise one or more anti-cancer agents (e.g., chemotherapeutic agents). The pharmaceutical formulations of the invention can also comprise at least one hypermutated cancer gene product or hypermutated cancer gene expression-inhibition compound (or at least one nucleic acid comprising sequences encoding them), which are encapsulated by liposomes and a pharmaceutically-acceptable carrier. Especially suitable pharmaceutically-acceptable carriers are water, buffered water, normal saline, 0.4% saline, 0.3% glycine, hyaluronic acid and the like.

In a particular embodiment, the pharmaceutical compositions of the invention comprise at least one hypermutated cancer gene product or hypermutated cancer gene expression-inhibition compound (or at least one nucleic acid comprising sequences encoding them) that is resistant to degradation by nucleases. One skilled in the art can readily synthesize nucleic acids that are nuclease resistant, for example, by incorporating one or more ribonucleotides that is modified at the 2′-position into the hypermutated cancer gene product. Suitable 2′-modified ribonucleotides include those modified at the 2′-position with fluoro, amino, alkyl, alkoxy, and O-allyl.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one hypermutated cancer gene product or hypermutated cancer gene expression-inhibition compound (or at least one nucleic acid comprising sequences encoding them). A pharmaceutical composition for aerosol (inhalational) administration can comprise 0.01-20% by weight, preferably 1%-10% by weight, of the at least one hypermutated cancer gene product or hypermutated cancer gene expression-inhibition compound (or at least one nucleic acid comprising sequences encoding them) encapsulated in a liposome as described above, and a propellant. A carrier can also be included as desired; e.g., lecithin for intranasal delivery.

The pharmaceutical compositions of the invention can further comprise one or more anti-cancer agents. In a particular embodiment, the compositions comprise at least one hypermutated cancer gene product or hypermutated cancer gene expression-inhibition compound (or at least one nucleic acid comprising sequences encoding them) and at least one chemotherapeutic agent. Chemotherapeutic agents that are suitable for the methods of the invention include, but are not limited to, DNA-alkylating agents, anti-tumor antibiotic agents, anti-metabolic agents, tubulin stabilizing agents, tubulin destabilizing agents, hormone antagonist agents, topoisomerase inhibitors, protein kinase inhibitors, HMG-CoA inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial and exotoxic agents. Examples of suitable agents for the compositions of the present invention include, but are not limited to, cytidine arabinoside, methotrexate, vincristine, etoposide (VP-16), doxorubicin (adriamycin), cisplatin (CDDP), dexamethasone, arglabin, cyclophosphamide, sarcolysin, methylnitrosourea, fluorouracil, 5-fluorouracil (5FU), vinblastine, camptothecin, actinomycin-D, mitomycin C, hydrogen peroxide, oxaliplatin, irinotecan, topotecan, leucovorin, carmustine, streptozocin, CPT-11, taxol, tamoxifen, dacarbazine, rituximab, daunorubicin, 1-β-D-arabinofuranosylcytosine, imatinib, fludarabine, docetaxel, FOLFOX4.

The invention also encompasses methods of identifying an inhibitor of tumorigenesis, comprising providing a test agent to a cell and measuring the level of at least one hypermutated cancer gene product in the cell. In one embodiment, the method comprises providing a test agent to a cell and measuring the level of at least one hypermutated cancer gene product associated with DNA mutations in cancer cells. An increase in the level of the hypermutated cancer gene product in the cell after the agent is provided, relative to a suitable control cell (e.g., agent is not provided), is indicative of the test agent being an inhibitor of tumorigenesis In other embodiments the method comprises providing a test agent to a cell and measuring the level of at least one hypermutated cancer gene product associated with DNA mutations in cancer cells. A change in the level of the hypermutated cancer gene product in the cell after the agent is provided, relative to a suitable control cell (e.g., agent is not provided), is indicative of the test agent being an inhibitor of tumorigenesis. In a particular embodiment, at least one hypermutated cancer gene product associated with alered genetic structure in cancer cells is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456.

Suitable agents include, but are not limited to drugs (e.g., small molecules, peptides), and biological macromolecules (e.g., proteins, nucleic acids). The agent can be produced recombinantly, synthetically, or it may be isolated (i.e., purified) from a natural source. Various methods for providing such agents to a cell (e.g., transfection) are well known in the art, and several of such methods are described hereinabove. Methods for detecting the expression of at least one hypermutated cancer gene product (e.g., Northern blotting, in situ hybridization, RT-PCR, expression profiling) are also well known in the art. Several of these methods are also described hereinabove.

The invention will now be illustrated by the following non-limiting examples.

The following Materials and Methods were used in the Examples:

Databases and algorithms. The dbEST database (Release 23 Jul. 2004), contained more than 5.6 million human ESTs (exceeding 3,009 million nucleotides in length). Library annotations were used to subdivide ESTs in cancer and control. Coding sequences were extracted from Human Genome RefSeq mRNA database (27,184 sequences) and aligned against the ESTs by using BLAST. Total number of analyzed nucleotides corresponding to coding regions was 792,814,405 in cancer libraries and 586,806,978 in control libraries. Perl and Bioperl were used to develop all the scripts and implement the system. BLAST was set to recover up to 500 alignments for each query. A “cancer mismatches” SQL database was populated with a total of 43,965,904 mismatches and gaps extracted from 3,839,543 alignments. The mismatches in the first or last ten nucleotides of the alignments were not considered for subsequent statistical analysis. A candidate mutation was considered only once for each dbEST library, to avoid bias due to RNA copy number. Statistics for amino acid substitutions, synonymous nucleotide substitution and frame-shifts were calculated for each human coding sequence. The 8,972 genes with highest variability in the number of mismatches (IQR>0.5) were retained for cancer testing.

P-value, q value and false detection rate calculation. Procedures were devised for calculation of gene-specific p-values and false detection rates in each one of the three described tests. Bootstrap analysis was used to compute the adjust probability that a human gene was affected in cancer but not in normal ESTs (paired T test based procedures). The resampling test allowed to define confidence limits for each different gene and to effectively tackle local issues such as DNA composition, CpG occurrence, and protein length. The resampling procedure was performed only on the protein residues found to be above T threshold. For 1000 cycles the ESTs belonging to cancer and normal classes were randomly sampled with replacement, to form two simulated classes with the same size as the original ones. The gene specific p-values were represented by the frequency at which the resampling test scored equal or above that of the respective test. Null p-values were set to half of the lowest p-value in the whole simulation. A range of simulations were performed to choose the lowest number of resampling cycles yielding stable p-values through a short gene list and 1000 cycles were found to be the minimum requirement. An identical procedure was used to calculate false positive detection rates (FDRs), with the exception that also a random measure called the real measure was derived from the first resampling cycle. The overall frequency of the genes with equal or lower p-values then corresponded to the false detection rate (pFDR). The q-values (Storey, J., D. and Tibshirani, R. Proc. Natl. Acad. Sci. USA, 100: 9440-9445 (2003)) were also calculated, by using QVALUE package, which uses the list of p-values resulting from the simultaneous testing of 8972 human genes and estimates their q-values. The robust bootstrap method was used to calculate pi_{—}0 when estimating no, therefore the q-value estimate is a direct finite sample estimate of pFDR. There was essentially very strong agreement between the calculated FDRs and the q-values for the cancer genes.

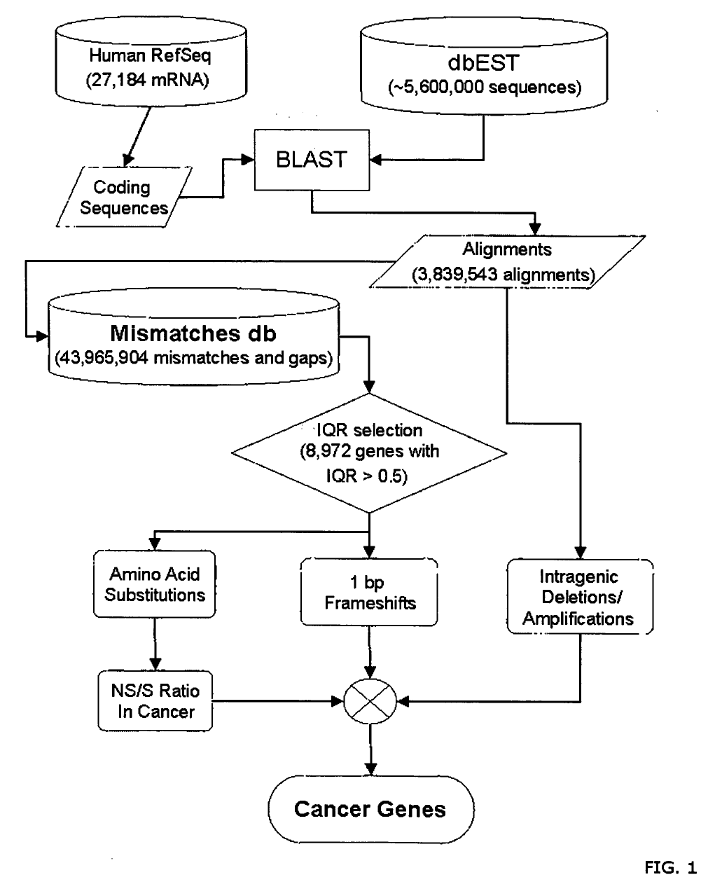

FIG. 1 shows a flow diagram shows the overall procedure for discovery of the mutated human genes in cancer. Since ESTs are single pass sequences, they often contain sequencing errors. To attenuate this problem the procedure retrieved candidate mutations only in the region of maximum nucleotide identity to the query. Notwithstanding this, the main assumption was that equal sequencing error rate is present in the two dbEST populations, those derived from control and those from cancer cells. Therefore, after normalization, the sequencing errors are essentially balanced out when the cancer and control comparison is performed by t test over a large enough sliding window. The p-values were calculated by using bootstrap analysis. To detect point mutations associated to library haplotypes rather than to mRNA copies each mismatch was recorded only once for each different library. Nevertheless no difference in the p-values for the most significant genes was apparent when this “haplotype” filter was disabled. The test for NS to S ratio, measuring selective pressure for amino acid mutation, should be relatively resilient to presence of sequencing errors and differential expression and was implemented to identify causal mutations. The deletions/amplifications were measured by an in-silico array CGH-like method.

### EXAMPLE 1

### Identification of Genes with High Frequency of Amino Acid Substitutions in Cancer

Statistics. In the first of three different measures relating to point mutations, the frequency of amino acid substitution over a 25-codons window was compared for each gene in normal and cancerous tissues by using paired T test. Normalization of mismatches for the control and cancer classes was attained by using a gene specific local normalization factor. The factor was derived by dividing the respective counts of ESTs in both classes at each nucleotide position of the query. The score assigned to each human RefSeq gene corresponded to the sum of the T scores values exceeding the confidence limit for p<0.05 over the sliding window (i.e. the area of the peak in the graph above the threshold). P-values (pSubs) were obtained by using bootstrap analysis, randomly selecting, 1000 times, ESTs derived from cancer and normal tissues.

The following point mutation test consisted in the evaluation of the selective pressure for amino acids changes in genes encoding cancer proteins. This filter was implemented in order to separate causal from bystander mutations. The ratios of non-synonymous (NS) to synonymous (S) nucleotide substitutions within the cancer and normal ESTs were calculated for each RefSeq entry. A paired T-test was used to compare the cancer and normal NS/S substitution ratios at different codons in each RefSeq entry. When the number of synonymous substitutions at denominator was null, unity was added to both numerator and denominator. Only the amino acid positions found to be significant for amino acid substitutions (in the first approach) were evaluated here. The gene specific p-values (pNSSR) and FDR for the NS to S comparison were again obtained by bootstrap, randomly selecting ESTs derived from cancer and normal tissues.

Results. Different statistical tests were combined to identify the genes which are often and extensively mutated in cancer with the purpose of reducing the number of false positives. Starting from a complete human mRNA repository, 27,184 coding sequences were aligned to more than 5.6 million human EST sequences. BLAST produced successful alignments for 24,932 human open reading frames and 33,614,754 mismatches were recorded in a cancer mismatches database. 792,814,405 nucleotides were analyzed corresponding to coding regions for the cancer class and 586,806,978 for the control. ESTs are present in cDNA libraries proportionally to the expression of the respective genes. Thus the abundance of a gene was defined as the relative frequency of its analyzed nucleotides in that class. Gene specific fold changes in cancer vs. control were calculated by dividing the respective abundances in the two classes. Filtering was then performed to pass only genes which had a high number of mismatches irrespective of cancer status. Inter-quantile range (IQR) was therefore applied and 8,972 genes with IQR larger than 0.5 were retained for further analysis.

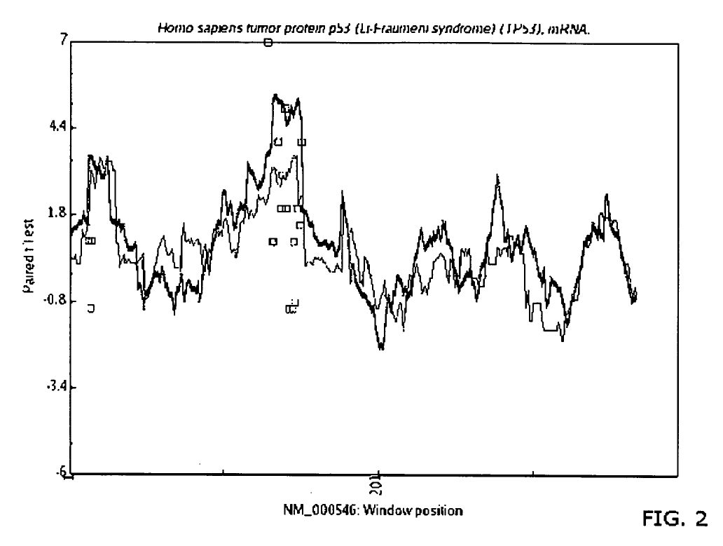

When looking for frequent amino acid substitutions, after normalization, a paired T test was applied to sliding windows of 25 codons. To perform robust tests a codon in the test was considered only when aligned at least 10 times in each class, control and cancer. This technique was chosen to filter noise, i.e. sequencing errors, and to select genes with short range clustering of frequent mutations. For each gene a p-value (pSubs) was then associated to the area of the peaks composed by the significant t scores. The p-value was calculated by bootstrap analysis. As an example, the plot of the T scores for each 25-codons window in the coding region of TP53 cancer gene is shown in FIG. 2. The region of Aa substitutions well overlaps with the TP53 DNA binding domain, which is mutated most frequently in human cancer. Bootstrap was then used again to compute the genome-wide false positive detection rate (pFDR) resulting by multiple testing and considered the number of proteins above threshold as the number of false positives present in the identified cancer genes.

### EXAMPLE 2

### Identification of Genes with High Frequency of Frame-Shift Mutations in Cancer Cells

Statistics: In the second example, a T-test based procedure similar to that of Example 1 was applied to the number of frame-shifts induced by 1 nucleotide insertions or deletions. Longer DNA alterations were not recorded, as they were very rare. Frame-shifts of one base will generally produce premature protein termination or other major differences in primary structure.

Results: A bias could be present in the measures described in Example 1 due to passenger mutations produced by altered DNA repair systems in some cancer cells. For this reason an unbiased test was additionally implemented, using the ratio of non-synonymous (NS) to synonymous (S) DNA mutations. This represents a measure of the selective mutation pressure during tumour progression, as synonymous alterations are unlikely to exert a growth advantage and will be selectively lost (Samuels, Y. et al. Science 304:554 (2004)). The codons significant for amino acid substitutions (p<0.05) were evaluated with this test to generate the relative gene specific probability (pNSSR). The NS/S ratios in the TP53 mutated region were found to be higher than in the normal tissues by paired T test (p<0.033, FDR=0.092) and are shown in graph of FIG. 2. The probability of a protein having frequent amino acid changes coupled with selective positive pressure in cancer, pAa, events which are not independent, was defined as the average of the respective pSubs and pNSSR p-values.

FIG. 2 shows Profile of TP53 point mutation analysis in human cancer ESTs. When applied to TP53 each one of the three cancer mutation tests yielded significant p-values. The p-value for amino acid substitution (pSubs) was 0.022, while the ratio of NS to S mismatches over the mutated region was found to be significantly higher than in normal tissues (pNNSR=0.031, FDR=0.092). Only one hundred and ninety one transcripts had a p-value for 1 bp frame-shifts lower or equal to that of TP53 (p-value=0.0005 and FDR=0.047). The thick line shows the paired T score for amino acid mutations in cancer vs. normal ESTs, while the thin line shows the paired T score for 1 nucleotide frameshifts. The open squares show the difference between the NS/S ratio in cancer and in normal ESTs. It is apparent that there are higher absolute value NS/S ratios in cancer. In fact positive NS/S differences (black) are higher in number and larger in absolute value than the negative ones (gray). Gene specific confidence limits for the paired T test were calculated by bootstrap analysis. Two classes were composed by random extracting 1000 times with replacement cancer or normal status from the library classes (Efron and Tibshirani, 1993; Davison et al, 1997).

### EXAMPLE 3

### Identification of Genes with High Frequency of Partial Gene Deletion/Amplification in Cancer Cells

Data Mining The frequency at each nucleotide position of each open reading frame in the cancer and control libraries was then used to perform an in-silico ECGH (expression CGH like), where the nucleotide frequencies were assimilated to the fluorescence intensity. Conditions required for the CGH test were of a minimal intensity of in each of the classes. These conditions ensured that robust measures were considered. The gene score corresponded to the number of significant 1092 intensity ratios (CL were mean +−2×SD, i.e. −0.02+−2×0.37). The standard deviation and mean for 1092 intensity ratios were calculated on all the genes, after removing 2000 of those scoring highest for point mutations in the previous tests. In this distribution median and mean were identical. Genes were ranked according to the scores.

Results: Additional distortion in the system is introduced by relative abundance of mRNA levels which change in cancer and control tissues. Although normalization was carried out, genes that are over-expressed in cancer are still more likely to present “candidate cancer amino acid substitutions” and vice-versa. Thus a penalty was included for those genes which are more transcribed in cancer libraries by multiplying each gene pAa and pFS by the relative probability of being present in a cancer tissue. This probability, pCancer, was defined as the relative abundance in cancer divided by the sum of the relative abundance in the two classes. The probability of a protein having cancer specific amino acid substitutions and frame-shifts, two independent events, was defined as the product of the respective corrected p-values (pAa×pCancer×pFS×pCancer). Thus the p-value threshold for cancer mutation significance becomes 2.5×10⁻⁵ (corresponding to a p-value of 0.01 in each one of three tests and an equal expression in cancer and control tissues). In the point mutation tests, TP53 and PTEN ranked 236 and 242 respectively (pFDR=0.0035, p-value<7×10⁻⁶). The q-value was used to measures the proportion of false positives incurred (called the false discovery rate) in the cancer point mutation test (Storey and Tibshirani, 2003). Overall 361 different human transcripts had p-values lower than 2.5×10⁻⁵ with q-values<=0.0026. Some oncogenes like for example ErbB2 were also present among this point mutated gene set. ErbB2 was recently reported to be frequently mutated in lung cancer (Stevens et al, 2004).

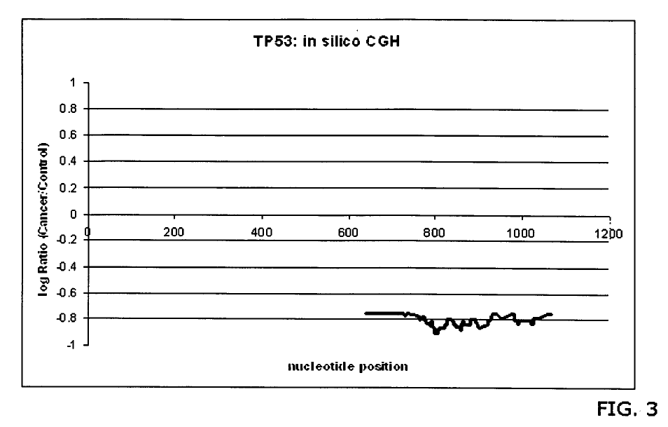

The identification of clusters of point mutations in the EST database is severely affected by many issues as described above. Thus another independent criterion was added for cancer gene selection, based on gene deletions and amplifications. To this end an in-silico ECGH (expression CGH) experiment was devised and performed, by measuring the relative frequencies of every mRNA position in cancer and control ESTs. By analogy to array CGH, each DNA position in an open reading frame can be considered as a probe and its frequency in the cancer and controls can be assimilated to the intensities of the two fluorescence channels. The absolute frequency within a cDNA clone population can be variable, being a function of the cloning procedure and it is generally higher towards the 3′ end of the messenger RNA. Nevertheless the ratio of the frequencies at each position in the two classes should be constant. In principle, after normalization, the frequency ratios should be close to 1, if no deletions or amplifications are encountered. In the test an exon position was considered only when its frequency was at least of 10 in each of the classes. This filter ensured that low intensity signals were excluded and robust measures could be produced. The in-silico ECGH procedure should be unaffected by the sequencing errors present in the ESTs since it is based on the presence of an exon element rather than the base called at that position. Thus this method well complements those used above for point mutations. The “in silico ECGH” profile for TP53 is shown in FIG. 3. For each gene the log ratios were scored when distant more than two standard deviations from the mean. The standard deviation and mean were calculated on all the genes, after removing those most significant for point mutations. Different Isoforms often appear to be selected by ECGH in similar protein regions (i.e. DDX1, DDX21, TRIM, MCM). Since the homology level is below BLAST stringency this results can not be ascribed to sequence conservation, rather are common mechanisms associated to cancer. This ECGH method though can not technically distinguish genomic rearrangements from differential exon usage in cancer and normal tissues. For example, The two variants of heterogeneous nuclear ribonucleoprotein A2/B1 (HNRPA2B1) could be detected. The transcript variant B1 and A2 differ from 12 Aa at the amino-terminus and precisely these were identified by the algorithm. Furthermore C-terminus variants can also be detected (i.e. PSMD4 proteasome (prosome, macropain) 26S subunit, non-ATPase, 4). On the other hand most often the detected alteration does not map to any alternative exon or splicing variant. Overall the ECGH ranking is still highly relevant to cancer since both detected putative genomic rearrangement and/or alternative exon usage are associated to functional cancer protein variants.

### EXAMPLE 4

### Identification of Hypermutated Cancer Genes with High Frequency of Overall Mutations and Genomic Structural Alterations in Cancer Cells

Statistics: A final list of the hypermutated cancer genes was compiled by averaging the rank orders of each one of the three tests: amino acid substitution, single nucleotide frame-shift, partial gene deletion/amplification.

Results: PTEN and TP53 ranked respectively 96 and 130 best among the human transcripts, indicating a success of our technique in enriching for cancer genes. The top ranking genes are reported in Table I. While both PTEN and TP53 recessive cancer genes increase their ranks after combining point mutation selection with the in silico CGH analysis, other dominant cancer genes, like for example ErbB2 decrease their standings. This might reflect the propensity of the in silico CGH test to identify genes which are inactivated by partial gene rearrangements. Interestingly protein kinases are present in the expected frequency by random association (only 4 in the top 185 ranking genes).

For each putative cancer gene, the altered coding regions were then pinpointed. To this purpose gene specific confidence limits for both amino acid substitution and deletions/insertions (p<0.05) were calculated by using bootstrap. These mRNA segments thus define the minimal region of human transcriptome which is mutated in cancer

The molecular functions, biological processes and cellular compartments associated with the cancer mutated genes were statistically evaluated using GOAL (Volinia, S. et al. Nucleic Acids Res. 32:W492-9 (2004)). Most of the functions and processes identified are known to be involved in cancer pathogenesis, such as apoptosis, regulation of cell cycle, cell adhesion, chromatin, protein phosphorylation, dephosphorylation and ubiquitination. In parallel, gene networks of mutated genes were constructed by means of automated literature data mining. PubGene was used to assess the hypothesis that the mutated mRNAs included coherent groupings. When mutated cancer genes were analysed, 145 significant gene networks (score>0.718, p-value<0.01) were identified. To assess these results the PubGene analysis was repeated on each of ten random gene selections. In this simulation the number of significant gene networks (i.e. with p-value<0.01) was never higher than 27, (mean=18.3, standard deviation=8.47); therefore the presence of 145 gene networks in the identified cancer genes reveals bona-fide relations (p-value<0.001).

The possibility that the mismatches identified here might correspond to rare polymorphisms rather than mutations was evaluated. Recently, Qiu and co-workers (2004) measured SNP-tumour association in the EST database. Their analysis was restricted to mutations described in the SNP database. They identified 327 SNPs more frequent in tumours and which induced amino acid substitution. Interestingly, neither landmark cancer genes, such as TP53 and PTEN, nor any of the genes identified here are present within their significant ESTs. However that is not a formal demonstration that the genes identified correspond to somatic mutation targets, although TP53 is evidence in favour of this hypothesis. Furthermore it is possible that some of the detected mutations are germine and constitute genetically predisposed background to cancer insurgence. Finally our results for the first time indicate that data from genome sequencing can be used for systematically detect partial gene deletions/amplifications.

The relevant teachings of all publications cited herein that have not explicitly been incorporated by reference, are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of diagnosing whether a subject has, or is at risk for developing, a cancer, comprising determining the nucleotide sequence or the genomic structure of at least one hypermutated cancer gene product in a test sample from the subject, wherein an alteration in the nucleotide sequence or the genomic structure of the hypermutated cancer gene product in the test sample, relative to the nucleotide sequence or the genomic structure of a corresponding hypermutated cancer gene product in a control sample, is indicative of the subject either having, or being at risk for developing, a cancer.

2. The method of claim 1, wherein the at least one hypermutated cancer gene product is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247 and combinations thereof.

3. The method of claim 1, wherein the at least one hypermutated cancer gene product is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}00681.9, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

4. A method of diagnosing whether a subject has, or is at risk for developing, a cancer, comprising: (1) labeling DNA or RNA from a test sample obtained from the subject to provide a set of target oligodeoxynucleotides; (2) hybridizing the target oligodeoxynucleotides to a microarray comprising hypermutated cancer gene-specific probe oligonucleotides to provide a hybridization profile for the test sample; and (3) comparing the test sample hybridization profile to a hybridization profile generated from a control sample, wherein an alteration in the signal of at least one hypermutated cancer gene is indicative of the subject either having, or being at risk for developing, a cancer.

5. The method of claim 4, wherein the signal of at least one hypermutated cancer gene, relative to the signal generated from the control sample, is decreased.

6. The method of claim 4, wherein the signal of at least one hypermutated cancer gene, relative to the signal generated from the control sample, is increased.

7. The method of claim 4, wherein the microarray comprises hypermutated cancer gene-specific probe oligonucleotides for one or more hypermutated cancer genes selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}1139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

8. A method of inhibiting tumorigenesis in a subject who has, or is suspected of
having, a cancer in which at least one hypermutated cancer gene product is down-regulated or up-regulated in the cancer cells of the subject, relative to control cells,
comprising: (1) when the at least one hypermutated cancer gene product or activity is down-regulated in the cancer cells, administering to the subject an effective amount of at least one isolated hypermutated cancer gene product, or an isolated variant or biologically-active fragment thereof, such that tumorigenesis is inhibited in the subject; or (2) when the at least hypermutated cancer gene product or activity is up-regulated in the cancer cells, administering to the subject an effective amount of at least one compound for inhibiting expression of the at least one hypermutated cancer gene product, such that tumorigenesis is inhibited in the subject.

9. The method of claim 8, wherein the at least one isolated hypermutated cancer gene product is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

10. A method of inhibiting tumorigenesis in a subject who has a cancer, comprising: (1) determining the nucleotide sequence and genomic structure of at least one hypermutated cancer gene product in cancer cells from the subject, relative to control cells; and (2) altering the amount of hypermutated cancer gene product expressed in the cancer cells by: (i) administering to the subject an effective amount of at least one isolated hypermutated cancer gene product, or an isolated variant or biologically-active fragment thereof, if the amount of the hypermutated cancer gene product or activity expressed in the cancer cells is less than the amount of the hypermutated cancer gene product or activity expressed in control cells; or (ii) administering to the subject an effective amount of at least one compound for inhibiting expression or activity of the at least one hypermutated cancer gene product, if the amount of the hypermutated cancer gene product or activity expressed in the cancer cells is greater than the amount of the hypermutated cancer gene product or activity expressed in control cells, such that tumorigenesis is inhibited in the subject.

11. The method of claim 10, wherein the at least one hypermutated cancer gene product is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

12. A pharmaceutical composition for treating a cancer, comprising at least one isolated hypermutated cancer gene product, or an isolated variant or biologically-active fragment thereof, and a pharmaceutically-acceptable carrier.

13. The pharmaceutical composition of claim 12, wherein the at least one isolated hypermutated cancer gene product corresponds to a hypermutated cancer gene product or activity that is altered in cancer cells relative to suitable control cells.

14. A pharmaceutical composition for treating a cancer, comprising at least one hypermutated cancer gene product activity-inhibition compound and a pharmaceutically-acceptable carrier.

15. The pharmaceutical composition of claim 14, wherein the at least one hypermutated cancer gene product activity-inhibition compound is specific for a hypermutated cancer gene product that is altered in cancer cells relative to suitable control cells.

16. The pharmaceutical composition of claim 15, wherein the at least one hypermutated cancer gene product activity-inhibition compound is specific for a hypermutated cancer gene product selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.

17. A method of identifying an inhibitor of tumorigenesis, comprising providing a test agent to a cell and determining the nucleotide sequence and gene structure of at least one hypermutated cancer gene product associated with cancers, wherein an alteration in the hypermutated cancer gene activity in the cell, relative to a suitable control cell, is indicative of the test agent being an inhibitor of tumorigenesis.

18. The method of claim 17, wherein the hypermutated cancer gene product is selected from the group consisting of NM_{—}001273, NM_{—}080921, NM_{—}014865, NM_{—}003072, NM_{—}004104, NM_{—}004990, NM_{—}004599, NM_{—}013417, NM_{—}172230, NM_{—}148842, NM_{—}021948, NM_{—}014014, NM_{—}001417, NM_{—}002271, NM_{—}005030, NM_{—}182917, NM_{—}001923, NM_{—}005762, NM_{—}005348, NM_{—}001418, NM_{—}002266, NM_{—}012218, NM_{—}002466, NM_{—}005557, NM_{—}000691, NM_{—}001569, NM_{—}001090, NM_{—}201524, NM_{—}002291, NM_{—}002230, NM_{—}001605, NM_{—}017647, NM_{—}002541, NM_{—}005438, NM_{—}133645, XM_{—}290401, NM_{—}000968, NM_{—}144733, NM_{—}004741, NM_{—}020414, NM_{—}004793, NM_{—}000224, NM_{—}006819, XM_{—}379877, NM_{—}001436, NM_{—}004247, NM_{—}000967, NM_{—}199413, NM_{—}032044, NM_{—}013403, NM_{—}012469, NM_{—}003169, NM_{—}006470, NM_{—}021991, XM_{—}290506, NM_{—}153280, NM_{—}080686, NM_{—}000289, NM_{—}003875, NM_{—}024658, NM_{—}003074, NM_{—}152298, NM_{—}007126, NM_{—}139215, NM_{—}147200, XM_{—}290345, XM_{—}377464, NM_{—}021873, NM_{—}006429, NM_{—}015292, NM_{—}005956, NM_{—}001940, NM_{—}000526, NM_{—}001747, NM_{—}024311, NM_{—}003938, NM_{—}005336, NM_{—}003751, NM_{—}006839, NM_{—}000937, NM_{—}012112, NM_{—}006739, NM_{—}005916, NM_{—}138421, NM_{—}001379, NM_{—}006289, NM_{—}004939, NM_{—}001357, NM_{—}000422, NM_{—}002417, NM_{—}005968, NM_{—}003754, XM_{—}378197, NM_{—}002473, NM_{—}002972, NM_{—}000546, NM_{—}001102, XM_{—}374522, NM_{—}001458, NM_{—}172020, XM_{—}039701, NM_{—}000701, NM_{—}002298, NM_{—}015179, NM_{—}002967, NM_{—}018454, NM_{—}006796, NM_{—}005558, NM_{—}006812, NM_{—}003400, NM_{—}001034, NM_{—}004728, NM_{—}178313, NM_{—}145714, XM_{—}085722, NM_{—}001798, NM_{—}181054, NM_{—}001916, NM_{—}002668, NM_{—}052963, NM_{—}031243, NM_{—}002638, NM_{—}003146, NM_{—}005564, NM_{—}016292, XM_{—}375253, NM_{—}003815, NM_{—}001363, NM_{—}032271, NM_{—}000314, NM_{—}198830, NM_{—}032999, NM_{—}015935, NM_{—}134447, NM_{—}006928, NM_{—}014390, NM_{—}020117, NM_{—}001619, NM_{—}022820, NM_{—}005526, NM_{—}033500, NM_{—}001903, NM_{—}004844, NM_{—}006372, NM_{—}022743, NM_{—}007355, NM_{—}012426, NM_{—}000088, NM_{—}182926, NM_{—}004563, NM_{—}014612, NM_{—}004446, XM_{—}168585, NM_{—}014173, NM_{—}144596, NM_{—}138925, NM_{—}174889, NM_{—}006452, NM_{—}004689, NM_{—}015315, XM_{—}379904, NM_{—}014753, NM_{—}198309, NM_{—}002810, NM_{—}002388, NM_{—}014938, NM_{—}018031, NM_{—}023007, NM_{—}002362, NM_{—}006088, NM_{—}002014, NM_{—}003823, NM_{—}030877, NM_{—}003752, NM_{—}001456, NM_{—}006286, NM_{—}145685, NM_{—}003103, NM_{—}002265, NM_{—}005915, XM_{—}376630, NM_{—}006295, NM_{—}145810, NM_{—}015456 and combinations thereof.
